(19) United States
(12) Patent Application Publication
(10) Pub. No.: US 2015/0297099 A1
(43) Pub. Date: Oct. 22, 2015

(54) PARAMETRIC ELECTRIC IMPEDANCE TOMOGRAPHY OF THE CHEST

(71) Applicant: Ramot at Tel-Aviv University Ltd., Tel-Aviv (IL)

(72) Inventors: Shimon ARAD (ABBOUD), Tel-Aviv (IL); Muhammad MAHAJNA, Umm El Fahem (IL); Keren HORMAN, Petach-Tikva (IL)

(21) Appl. No.: 14/433,055

(22) PCT Filed: Oct. 3, 2013
(86) PCT No.: PCT/IL2013/050812

§ 371 Date: Apr. 2, 2015

Related U.S. Application Data
(60) Provisional application No. 61/709,169, filed on Oct. 3, 2012.

(51) Int. Cl. A61B 5/029; A61B 5/0205; A61B 5/00

(52) U.S. Cl. CPC A61B 5/029; A61B 5/6869; A61B 5/0205; A61B 5/7278; A61B 5/0536

(57) ABSTRACT

A method for determining values of a set of one or more geometric parameters of a patient's heart, the method comprising:
a) making impedance measurements of the patient's chest, at least some of the measurements using internal electrodes; and
b) finding values of the set of parameters that provide a good fit to results of the impedance measurements.

### RELATED APPLICATION/S

This application claims the benefit of priority under 35 USC §119(e) of U.S. Provisional Patent Application No. 61/709,169 filed Oct. 3, 2012, the contents of which are incorporated herein by reference in their entirety.

### FIELD AND BACKGROUND OF THE INVENTION

The present invention, in some embodiments thereof, relates to electric impedance tomography (EIT), and, more particularly, but not exclusively, to parametric EIT of the chest.

Cardiac stroke volume is an important quantity to measure in patients with heart disease. Impedance cardiography is a non-invasive technique for estimating stroke volume, typically using two external electrodes that each go around the patient's trunk, but it generally only provides relative values of stroke volume which can be compared over time for a given patient, rather than accurate absolute measures of stroke volume. Furthermore, impedance cardiography tends to be less accurate in patients with more severe heart disease, when it is most important to monitor stroke volume accurately. Impedance cardiography is described in the Wikipedia article at www.en.wikipedia.org/wiki/Impedance_cardiography. A major commercial producer of impedance cardiography devices is Cardiodynamics, owned by SonoSite since 2009. Their product line for impedance cardiography includes the BioZ® Dx system. Other producers of impedance cardiography devices include Cheetah Medical and NI Medical, both in Israel.

Several researchers have described systems for estimating cardiac stroke volume using electric impedance tomography (EIT), including Eyuboglu, B. M. et al., “In Vivo Imaging of Cardiac Related Impedance Changes,” March 1989, IEEE Engineering in Medicine and Biology Magazine, Vol. 8, pages 39-45; U.S. Pat. No. 5,311,878 and U.S. Pat. No. 5,746,214, both to Brown and Barber; D. C. Barber, Med. Phys., (1989), Vol. 16, pages 162-169; and Newell et al, “Phasic three-dimensional imaging of cardiac activity,” Physiol. Meas. 23, 203-209 (2002). These systems typically use 16 or even 32 electrodes arranged around the body, and do not produce accurate absolute estimates of stroke volume.

U.S. Pat. No. 6,134,472 to Strandberg et al describes the use of implanted pacemaker electrodes to estimate cardiac stroke volume. Only a relative measure of stroke volume as a function of heart rate is found, in order to optimize the heart rate at which a pacemaker is used.

Zlochiver et al, “Parametric EIT for monitoring cardiac stroke volume,” Physiol. Meas. 27, S139-S146 (2006) describes the use of parametric EIT for measuring cardiac stroke volume, using a two-dimensional model of the chest, using 8 external electrodes placed in a circle around the chest, and modeling the left ventricle as an ellipse with its major and minor diameter as free parameters. U.S. Pat. No. 8,131,354 to Arad (Abboud) describes a similar system for measuring cardiac stroke volume using electric impedance tomography modeling the left ventricle as an ellipse, and also describes using only 4 electrodes in a circle around the chest, and describes using a three-dimensional model of the chest and using more than one circle of electrodes around the chest, at different axial positions.

Cardiac patients often have lung edema, and monitoring lung edema is important in caring for cardiac patients. EVLW (Extra-Vascular Lung Water) can be monitored invasively using transpulmonary thermodilution. Commercial products that use this method include the PICCO device by Pulsion Medical Systems, and the EV1000 by Edwards Life Sciences.

Non-invasive monitoring of lung edema by electric impedance measurements has also been proposed. U.S. Pat. No. 5,749,369, and Charach, G. et al., “Transthoracic Monitoring of the Impedance of the Right Lung in Patients with Cardiogenic Pulmonary Edema,” Crit. Care Med. 2001, Vol. 29, No. 6, pages 1137-1144 discuss non-invasive systems using external electrodes to monitor lung edema. Published U.S. patent applications 2004/0102712 and 2005/0124908 to Belalcazar et al, and Belalcazar et al, “Improved lung edema monitoring with coronary vein pacing leads: a simulation study,” Physiol. Meas. 25, 475-487 (2004) describe methods of estimating lung edema using implanted pacemaker electrodes. These systems generally only make relative estimates of lung edema, to compare trends in the same patient over a period of time. Belalcazar et al have found that it is difficult to obtain even relative information about right lung edema from implanted pacemaker electrodes alone, but they say that such information can be obtained from additional external electrodes. U.S. Pat. No. 7,907,998 to Arad (Abboud) describes a system using implanted cardiac pacemaker electrodes that can independently measure left lung and right lung edema.

### SUMMARY OF THE INVENTION

An aspect of some embodiments of the invention concerns a parametric electric impedance tomography (pEIT) system for measuring lung edema and/or heart geometry, including cardiac stroke volume, by fitting a relatively small number of parameters of the heart and/or lungs to impedance measurements made with a relatively small number of internal or external electrodes, using an electrical model of the chest.

There is thus provided, in accordance with an embodiment of the invention, a method for determining values of a set of one or more geometric parameters of a patient's heart, the method comprising:
a) making impedance measurements of the patient's chest, at least some of the measurements using internal electrodes; and
b) finding values of the set of parameters that provide a good fit to results of the impedance measurements.

Optionally, the geometric parameters comprise parameters that determine a volume of the left ventricle, the method also comprising determining the volume of the left ventricle from the values of the set of parameters.

Optionally, the geometric parameters also comprise parameters that determine a position of the left ventricle.

Optionally, making the impedance measurements, finding the values of the set of parameters, and determining the volume of the left ventricle are done for each of a plurality of cardiac phases.

Optionally, the method also comprises finding the cardiac stroke volume from the volume of the left ventricle at each of the cardiac phases.

In an embodiment of the invention, finding the values of the set of parameters comprises:
a) assigning tentative values to the parameters;
b) calculating what results of the impedance measurements would be, using an electrical model of at least part of the patient's body, if the parameters had those values;
c) using the actual and calculated results of the impedance measurements, and a Jacobian of the calculated results with respect to the values of the parameters, to estimate new values of the parameters for which a measure of difference between the actual and calculated results would be smaller;
d) repeating (b) and (c) until values of the parameters are found that provide a sufficiently good fit to the results of the impedance measurements.

Optionally, the electrical model is a two-dimensional electrical model.

Alternatively, the electrical model is a three-dimensional electrical model.

Optionally, the parameters comprise three parameters corresponding to diameters of the left ventricle in three orthogonal directions, and the left ventricle is modeled in the three-dimensional model as a shape whose volume is determined by the three parameters.

Optionally, the electrical model is based on one or more images made of the patient's body.

Optionally, the internal electrodes comprise two electrodes implanted in the patient's heart.

Alternatively, the internal electrodes comprise three electrodes implanted in the patient's heart.

Optionally, said three electrodes are implanted respectively in the right atrium, in the right ventricle, and in the left ventricle or on a surface of the heart adjacent to the left ventricle, of the patient's heart.

Optionally, said two electrodes are implanted adjacent to and on different sides of the left ventricle.

Optionally, the internal electrodes comprise two electrodes implanted subcutaneously in the chest.

Optionally, the internal electrodes are comprised in an implanted therapeutic device.

Optionally, the implanted device is a member of the group consisting of a cardiac pacemaker, an implantable cardiovertor defibrillator, and a cardiac resynchronization therapy device.

Optionally, making impedance measurements of the chest comprises passing a current between a first two of the electrodes, while determining a voltage between a second two of the electrodes not used for passing current.

Optionally, the method also comprises finding a value of a specific impedance of the patient's lungs, or values of separate specific impedances of the patient's left lung and right lung, that provide a good fit to results of the impedance measurements.

Optionally, at least one of the impedance measurements is made using only external electrodes placed on the patient's skin, or using both internal electrodes and external electrodes placed on the patient's skin.

Optionally, at least one of the impedance measurements is made by passing a known current between a pair of internal electrodes while determining voltage between a pair of external electrodes, or by passing a known current between a pair of external electrodes while determining voltage between a pair of internal electrodes.

There is further provided, according to an embodiment of the invention, a method for determining one or more geometric parameters of a patient's heart, comprising:
a) making impedance measurements of the patient's chest using a total of 5 or fewer electrodes; and
b) finding values of the one or more geometric parameters that provide a good fit to results of the impedance measurements, according to a three-dimensional model of at least part of the patient's body.

Optionally, the geometric parameters comprise one or more parameters of the patient's left ventricle.

Optionally, the geometric parameters comprise dimensions of the left ventricle in three directions that are not coplanar.

Optionally, the method comprises finding the volume of the left ventricle from the parameters of the left ventricle.

Optionally, the impedance measurements are made, and the values of the parameters of the left ventricle are found, for a plurality of cardiac phases, and the volume of the left ventricle is found as a function of cardiac phase from the parameters of the left ventricle.

Optionally, the three-dimensional model models the left ventricle as a cup-like shape with the parameters of the left ventricle as free parameters that are fitted to the results of the impedance measurements.

Optionally, the cup-like shape is a truncated ellipsoid or a truncated elliptic paraboloid.

Optionally, the cup-like shape is a semi-ellipsoid.

Optionally, the impedance measurements are made using internal electrodes.

There is further provided, according to an exemplary embodiment of the invention, a method for determining one or more geometric parameters of a patient's heart, as a function of cardiac phase, the method comprising:
a) at each of a plurality of cardiac phases, making impedance measurements of the patient's chest; and
b) for each of the cardiac phases, finding values of the one or more geometric parameters that provide a good fit to results of the impedance measurements, according to an electrical model of at least part of the patient's body;
   wherein the electrical model includes the geometric parameters as free parameters in modeling the heart, models at least one tissue outside the heart as having one or more of a shape, a position, and a specific impedance that varies as a fixed function of cardiac phase.

Optionally, the electrical model models at least one of the tissues outside the heart as having a specific impedance that is lower at a cardiac phase for which the blood perfusion of the tissue is greater.

Optionally, the electrical model models at least one of the tissues outside the heart as having a position, shape, or both, that changes in response to being pushed or pulled directly or indirectly by a changing shape of the heart at different cardiac phases.

Optionally, the geometric parameters of the heart are parameters of the left ventricle usable for determining the volume of the left ventricle, and the method also comprises finding a volume of the left ventricle, from the values of said parameters, for each of the cardiac phases.

Optionally, the impedance measurements are made using internal electrodes.

There if further provided, according to an exemplary embodiment of the invention, a method for determining one or more geometric parameters of a patient's heart, the method comprising:
a) making impedance measurements of the patient's chest;
b) finding a set of one or more specific impedance values of the patient's lungs; and
c) finding values of the one or more geometric parameters of the heart that provide a good fit to data of the impedance measurements, according to an electrical model of at least part of the patient's body, with the specific impedance values of the lungs modeled as having the values found in (b).

Optionally, finding a set of one or more specific impedance values of the patient's lungs comprises measuring or estimating a degree of extra-vascular lung water and estimating one or more specific impedance values of the lungs, from the degree of extra-vascular lung water.

Optionally, the set of specific impedances of the lungs consists of a specific impedance of the left lung and a specific impedance of the right lung.

Optionally, finding the specific impedance values of the lungs comprises finding the specific impedance values that provide a good fit to the impedance measurements, according to an electrical model of at least part of the patient's body, the same as or a different model from the model used to find values of the geometric parameters of the heart.

Optionally, finding the set of specific impedance values of the lungs is done with a first electrical model in which the geometric parameters of the heart are not free parameters, and finding the values of the geometric parameters of the heart is done using a second electrical model in which the set of specific impedance values of the lungs are not free parameters, but are fixed at the values found using the first electrical model.

Optionally, finding the set of specific impedance values of the lungs and finding the set of geometric parameters of the heart are done using a same model in which the set of specific impedance values of the lungs and the set of geometric parameters of the heart are all free parameters that are varied to find a good fit to data of the impedance measurements.

Optionally, the geometric parameters of the heart are parameters of the left ventricle usable for determining the volume of the left ventricle, and the method also comprises finding a volume of the left ventricle, from the values of said parameters.

Optionally, making impedance measurements of the chest, and finding values of the geometric parameters of the heart, are done for a plurality of cardiac phases.

Optionally, the geometric parameters of the heart are parameters of the left ventricle usable for determining the volume of the left ventricle, and the method also comprises finding a volume of the left ventricle, from the values of said parameters, as a function of cardiac phase.

Optionally, finding the set of specific impedance values of the lungs comprises finding only a single set of impedance values at one cardiac phase or averaged over cardiac phases, and said set of specific impedance values of the lungs is used for each of the cardiac phases in the electrical model used for finding the geometric parameters of the heart.

Optionally, finding the set of specific impedance values of the lungs is done for each of the plurality of cardiac phases, and the set of specific impedance values of the lungs found for each cardiac phase is used in the electrical model for finding the geometric parameters of the heart for that cardiac phase.

Optionally, the impedance measurements are made using internal electrodes.

There is further provided, according to an embodiment of the invention, a method for determining specific impedance of a patient's lungs, comprising:
a) making impedance measurements of the patient's chest using at least two electrodes implanted in the heart, and at least two electrodes implanted outside the heart; and
b) finding a set of one or more specific impedance values of the lungs that provide a good fit to data of the impedance measurements, according to an electrical model of at least part of the patient's body.

Optionally, the implanted electrodes are electrodes of an implanted therapeutic device, and the electrodes implanted outside the heart are located on or adjacent to an implanted case of the therapeutic device.

Optionally, the set of one or more values of specific impedance of the lungs consists of a specific impedance of the left lung and a specific impedance of the right lung.

Optionally, at least one of the impedance measurements comprises passing a current between a first one of the electrodes outside the heart and a first one of the heart electrodes, while determining a voltage between a second one of the electrodes outside the heart and a second one of the heart electrodes that are not used for passing current.

Optionally, the at least two electrodes implanted in the heart comprise three electrodes implanted in the heart.

Optionally, the method also comprises finding values of the one or more geometric parameters of the heart that provide a good fit to data of the impedance measurements, according to the same or a different model of at least part of the patient's body, with the specific impedance values of the lungs modeled as having the values found.

Optionally, making impedance measurements of the chest, and finding values of the geometric parameters of the heart, are done for a plurality of cardiac phases.

There is further provided, according to an exemplary embodiment of the invention, a method of finding cardiac stroke volume of a patient using an impedance cardiography device, the method comprising:
a) finding a set of one or more specific impedance values of the patient's lungs;
b) obtaining a calibration of the effect of the specific impedance values of the lungs on the impedance cardiography device;
c) measuring cardiac stroke volume of the patient using the impedance cardiography device; and
d) applying the calibration to correct the cardiac stroke volume found by the impedance cardiography device.

Optionally, finding the one or more specific impedance values of the lungs comprises:
a) making impedance measurements of the patient's chest;
b) finding a set of one or more specific impedance values of the lungs that provide a good fit to data of the impedance measurements, according to an electrical model of at least part of the patient's body.

In an embodiment of the invention, obtaining a calibration comprises:
a) numerically solving Poisson's equation in an electric model of at least a portion of the patient's body including the heart and the lungs, for at least one value of cardiac stroke volume, for a systole and a diastole phase of the cardiac cycle, for the set of specific impedance values found for the patient's lungs, to find the impedances expected to be measured for that value of cardiac stroke volume, by the impedance cardiography device at the systole and diastole phases; and
b) comparing the impedances expected to be measured for that value of cardiac stroke volume and those values of specific impedance of the lungs, with impedances expected to be measured, for that value of cardiac stroke volume, for a patient with normal values of specific impedance of the lungs.

There is further provided, in accordance with an embodiment of the invention, a system for determining values of a set of one or more geometric parameters of a patient's heart, comprising:
a) a plurality of electrodes implanted in a patient's chest;
b) a power supply configured to pass a known current between a first pair of the electrodes through the chest;
c) a voltage sensor configured to measure a voltage between two of the electrodes, the same as or different from the first pair of electrodes, while the current is passing through the chest; and
d) a controller configured to find values of the set of parameters that provide a good fit to results of one or more measurements of voltage for known currents, according to an electrical model of at least part of the patient's body.

There is further provided, in accordance with an embodiment of the invention, a system for determining values of a set of one or more geometric parameters of a patient's heart, comprising:
a) a plurality of five or fewer electrodes;
b) a power supply configured to pass a known current between a first pair of the electrodes through the chest;
c) a voltage sensor configured to measure a voltage between two of the electrodes, the same as or different from the first pair of electrodes, while the current is passing through the chest; and
d) a controller configured to find values of the set of parameters that provide a good fit to results of one or more measurements of voltage for known currents, according to a three-dimensional model of at least part of the patient's body.

There if further provided, in accordance with an embodiment of the invention, a system for determining a set of one or more specific impedance values of a patient's lungs, comprising:
a) a plurality of electrodes comprising at least two electrodes implanted in the heart, and at least two electrodes implanted outside the heart;
b) a power supply configured to pass a known current between one of the electrodes outside the heart and one of the electrodes in the heart, through the chest;
c) a voltage sensor configured to measure a voltage between one of the electrodes outside the heart and one of the electrodes in the heart, while the current is passing through the chest; and
d) a controller configured to find the one or more specific impedance values of the lungs that provide a good fit to results of one or more measurements of voltage for known currents, according to an electrical model of at least part of the patient's body.

There is further provided, in accordance with an embodiment of the invention, a system for determining cardiac stroke volume of a patient, comprising:
a) an impedance cardiography device for measuring cardiac stroke volume;
b) a device for obtaining a set of at least an estimated value for one or more specific impedances of the patient's lungs; and
c) a controller that calculates a calibration of the impedance cardiography device for a given set of the one or more values of specific impedance of the patient's lungs.

Optionally, the method also comprises finding the cardiac stroke volume from the volume of the left ventricle at each of the cardiac phases.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic drawing of an implanted cardiac pacemaker, according to an exemplary embodiment of the invention;

FIG. 1B is a schematic drawing of an implanted cardiac pacemaker in a patient's chest, showing alternative locations for electrodes in the heart;

FIG. 2 is a block diagram schematically showing an EIT system comprising the implanted pacemaker of FIG. 1 together with an external controller and user interface, according to an exemplary embodiment of the invention;

FIGS. 3A, 3B and 3C are flowcharts showing methods of using the system shown in FIG. 2 to measure geometric parameters of the heart, or cardiac stroke volume, according to an exemplary embodiment of the invention;

FIG. 4 is a flowchart showing further details on finding specific impedance of the lungs when performing the method of FIG. 3;

FIG. 5 is a schematic drawing of a 2D model of the chest that can be used to find specific impedance of the lungs using the method of FIG. 4;

FIG. 6 is a flowchart showing further details on finding the left ventricle volume at different cardiac phases, when performing the method of FIG. 3;

FIG. 7 is a schematic drawing showing a 2D slice of the chest at different cardiac phases, that can be used for the method of FIG. 3;

FIG. 8 is a schematic drawing of a 2D model of the chest, that can be used for performing the method of claim 3; and

FIGS. 9A and 9B are schematic drawings showing alternative 3D models of the chest, that can be used for performing the methods of FIGS. 3A, 3B, and 3C.

FIG. 10A is a schematic drawing of a semi-ellipsoid that can be used to model the left ventricle, when performing the methods of FIGS. 3A-3C using a 3D model of the chest;

FIG. 10B shows the semi-ellipsoid of FIG. 10A overlapping the left ventricle; and

FIG. 11 is a plot of left ventricle volume vs. cardiac phase, obtained from performing the method of FIG. 3.

### DESCRIPTION OF SPECIFIC EMBODIMENTS OF THE INVENTION

The present invention, in some embodiments thereof, relates to electric impedance tomography (EIT), and, more particularly, but not exclusively, to parametric EIT of the chest.

An aspect of some embodiments of the invention concerns a method and system for making electric impedance measurements to determine geometric parameters of the heart, using at least some internal electrodes, for example electrodes of an implanted therapeutic device such as a pacemaker. The measurements can provide an absolute and precise measure of cardiac stroke volume, for example by using parametric EIT, in contrast to some prior art methods, for example that of Strandberg, cited above, which only provide a rough and relative estimate of cardiac stroke volume. Optionally, some impedance measurements also use external electrodes.

An aspect of some embodiments of the invention concerns a method and system for making electric impedance measurements with internal electrodes, using a model to determine free parameters of an organ or tissue, for example parameters of the heart.

As used herein, parametric EIT refers to a procedure in which an electrical model of at least part of the body is used to find an expected set of impedance data for a given set of values of a set of free parameters of the model, by solving the forward problem of Poisson's equation, and thence to solve the inverse problem, i.e. finding a set of values of the free parameters that will produce a set of expected impedance data that is a good fit to a set of measured impedance data. The free parameters can include, for example, specific impedance (averaged over a large region or specific to a small local region), size, shape, orientation, any combination of these quantities, and many other properties of different tissues and organs, that can be distinguished by impedance measurements. As used herein, making an impedance measurement means finding the ratio (including a possible phase factor e^{iφ}) of a voltage difference between a pair of electrodes, to current passing between a pair of electrodes, either the same pair electrodes that are exhibiting the voltage difference, or different pair of electrodes. Typically, for safety reasons, impedance in a living subject is measured by using a fixed value of current and measuring the voltage, and that is how impedance measurements will generally be described herein, but it should be understood that impedance measurements are also optionally made by holding the voltage fixed and measuring the current, or by adjusting the current to achieve a specified voltage, or by holding a combination of voltage and current fixed, and measuring a different combination of voltage and current. Although impedance measurements can be made while passing current between more than one pair of electrodes at a time, there is generally no new information to be gained by doing this, and impedance measurements will be described as measuring the voltage between a pair of electrodes while passing current between a pair of electrodes. The term “parametric EIT” is intended also to cover other model based reconstruction methods, which may be used to find one or more parameters. Such reconstruction methods need not be limited to using EIT, but could also use other types of reconstruction such as filtered back projection, and they need not be limited to using only a small number of electrodes.

Optionally, the system uses three electrodes implanted in the heart, for example in the right atrium, the right ventricle, and the left ventricle. Optionally, the system uses two implanted electrodes, not electrically connected to each other, implanted elsewhere in the chest, for example at the left pectoral muscle, for example associated with a case of an implanted therapeutic device. Optionally, the system is also used to make parametric EIT measurements of specific impedance of the lungs, optionally the left and right lungs separately. Such measurements of specific impedance of the lungs may be useful for diagnosing lung edema, which is often present in cardiac patients, and may provide an improved electrical model of the chest to use in finding the cardiac stroke volume. The parametric EIT method optionally uses an electrical model of the chest, for example a two-dimensional or three-dimensional model, with expected specific impedance values for other organs, muscles, and bones, optionally as a function of cardiac phase. Optionally, multiple two-dimensional models are used, one for each of a plurality of slices of the body. Optionally, the left ventricle is modeled as a simple geometric shape with a limited number of free parameters, for example as an ellipse in the two-dimensional case, or as a cup-like shape in the three-dimensional case, in order to find the left ventricle volume at different values of cardiac phase. Cardiac stroke volume is then found, from the example, from the difference between the maximum and minimum left ventricle volume over a cardiac cycle.

An aspect of some embodiments of the invention concerns using parametric EIT to find geometric parameters of the heart, for example parameters of the left ventricle, using a three-dimensional electrical model of the chest, using five or fewer electrodes. The parameters of the left ventricle can be used to find its volume, and to find the cardiac stroke volume, from the change in volume of the left ventricle over a cardiac cycle. Optionally the left ventricle is modeled as a cup-like volume of simple geometry, for example a semi-ellipsoid or a truncated elliptic paraboloid. The cup-like volume has only a few free parameters, for example its dimensions in three principal directions, the three spatial coordinates of its center of mass, and the three angles of its orientation in the body. Optionally, some of the free parameters, for example the angles of orientation, and/or the coordinates of the center of mass, are fixed at values found from an anatomical model of the chest, possibly as functions of the cardiac phase, but at least the dimensions in three principal directions are kept as free parameters for the parametric EIT procedure. Optionally, the impedance measurements are made using internal electrodes, for example three electrodes implanted in the heart, and two electrodes implanted elsewhere in the chest, for example in a pacemaker case. Alternatively, at least some external electrodes are used. However, the method, which uses realistic geometry and specific impedance values for the region around the heart, is especially useful when using implanted electrodes in the heart, which may be more sensitive to errors in the assumed configuration of tissues in and near the heart, than parametric EIT using external electrodes.

Optionally, the number of different impedance measurements that can be made is optionally at least as great as the number of free parameters, so that a least squares fit can be found for all the free parameters. The number of different impedance measurements that can be made may be, for example, the number of different pairs of electrodes that can be used for passing current, times the number of different pairs of electrodes that can be used for measuring voltage, optionally with different pairs being used for passing current and measuring voltage, in a given impedance measurement. However, not all such combinations of pairs of electrodes necessarily give much useful information, in every configuration, and not all such combinations are necessarily used for impedance measurements.

An aspect of some embodiments of the invention concerns measuring geometric parameters of any moving organ or tissue, for example the heart, for example to determine cardiac stroke volume, using a parametric EIT procedure, using an electrical model of the chest for which the geometric configuration and/or specific impedance of the tissues outside the heart vary with cardiac phase. For example, during the systole phase, when there is more blood perfusing the tissues of the chest, the specific impedance of those tissues is lower than during the diastole phase when there is less blood perfusing the tissues, and this is reflected in the model. Additionally or alternatively, the shapes and positions of various organs and other tissues may shift in response to the changing shape of the heart over a cardiac cycle, and/or due to changing degrees of blood perfusion. The model may be two-dimensional or three-dimensional, and may be based on the patient being examined, or on a typical or average patient.

An aspect of some embodiments of the invention concerns a method of measuring geometric parameters of the heart, for example to determine cardiac stroke volume, optionally using parametric EIT, in which a measurement is first made of specific impedance of the lungs, optionally the specific impedance of the left and right lungs independently. The values found for specific impedance of the lungs are then used in an electrical model of the patient's chest, when using parametric EIT to determine the geometric parameters of the heart. Optionally, the specific impedance of the lungs is itself determined from impedance measurements using parametric EIT. Alternatively, an invasive or non-invasive technique for evaluating extra-vascular lung water is used, for example transpulmonary thermal dilution, or a simple impedance method (not parametric EIT) calibrated to find a degree of fluid in the lungs. The specific impedance of the lungs may then be estimated from the degree of fluid in the lungs. This method has the potential advantage of yielding more accurate values for cardiac stroke volume, for example, than if a typical or average value of specific impedance of the lungs were assumed, because cardiac patients are likely to suffer from lung edema, in a degree that can vary greatly between different patients, and between the left and right lungs. Using parametric EIT is particularly useful for distinguishing left lung and right lung specific impedance, which is difficult to do using other electrical impedance methods, especially if only internal electrodes are used, as explained in U.S. Pat. No. 7,907,998 to Arad (Abboud). Because the lungs take up a large fraction of the volume of the chest, errors in the assumed specific impedance of the lungs can cause proportionately large errors in the cardiac stroke volume found using parametric EIT, and these errors can potentially reduced if the specific impedance of the lungs is accurately measured for that patient, and used in electrical model of the chest when the stroke volume is found. The geometry of the model may be either specific for that patient, or based on a typical or average patient, as described above, and either a two-dimensional or three-dimensional model may be used.

An aspect of some embodiments of the invention concerns using parametric EIT to measure specific impedance of the lungs, using internal electrodes, in which there are at least two electrodes located at different parts of the heart, and at least two electrodes, not electrically connected to each other, located elsewhere in the chest, for example on or near the case of an implanted therapeutic device. Using two different electrodes located away from the heart makes it possible to pass current from one electrodes not in the heart, to an electrode in the heart, while measuring voltage between the other electrode not in the heart, and the other electrode in the heart, and optionally this is done, avoiding the need to use the same electrodes for passing current and measuring voltage. This is potentially advantageous, because using the same electrode for passing current and measuring voltage can result in inaccurate measurements of voltage, if there is a substantial impedance in the interface between the electrode and the surrounding tissue, which can dominate the voltage measurement.

An aspect of some embodiments of the invention concerns a device for measuring cardiac stroke volume of a patient by an impedance cardiography device, in which a measurement or estimate is made of specific impedance of the lungs of the patient, and the impedance cardiography device is calibrated to correct for differences in the specific impedance of the lungs for that patient, and typical values for which the device is normally calibrated. Cardiac patients are likely to have different specific impedances of the lungs than most of the population, and different values for the left and right lungs, due to lung edema, and this can affect the results of cardiac stroke volume measurements using impedance cardiography. Optionally, the specific impedance is found separately for the left and right lungs. Optionally, the specific impedance of the lungs is measured by parametric EIT, for example using internal electrodes and/or external electrodes to make impedance measurements, and using a model of at least part of the body of the patient. Alternatively, the specific impedance of the lungs is estimated from measurements of extra-vascular lung water, for example invasive measurements using transpulmonary thermodilution. Alternatively, non-invasive measurements of extra-vascular lung water are used, for example simple impedance measurements of the chest, not parametric EIT, that have been calibrated to estimate extra-vascular lung water, for example using the methods described by Belalcazar et al in the paper and published patent applications cited above. Once a degree of extra-vascular lung water is known, an estimate can be made of the corresponding specific lung impedance, from first principles, from empirical studies, or from a combination of the two.

To calibrate an impedance cardiography device to take into account the specific impedance of a patient's lungs, the following exemplary procedure may be used. A numerical calculation is made solving Poisson's equation, using an electrical model of the body with the known specific impedances of the lungs, for two different values of cardiac phase, for example the diastole and systole phases, and taking into account the locations of the electrodes used by the device to inject current and measure voltage. The model includes the chambers of the heart, and the difference in size and shape of the left ventricle at systole and diastole. The expected measured impedance is found at the two cardiac phases, for a given cardiac stroke volume in the model, i.e. for a given difference in left ventricle volume between the two phases. The difference in impedance at the systole and diastole phases is likely to be linear with cardiac stroke volume, since it is relatively small difference, and in this way, the relation between change in impedance seen by the device, and cardiac stroke volume, can be found, for that value or values of specific impedance of the lungs.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

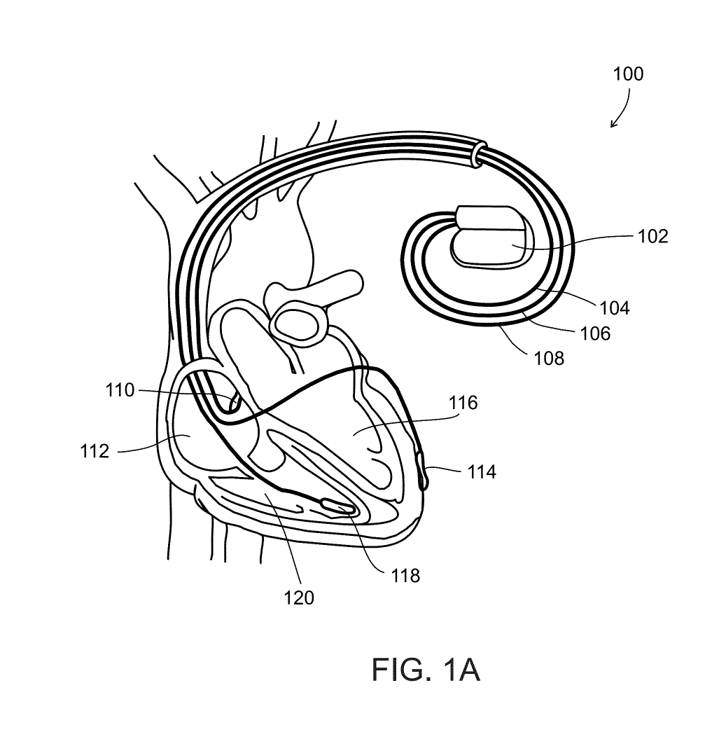

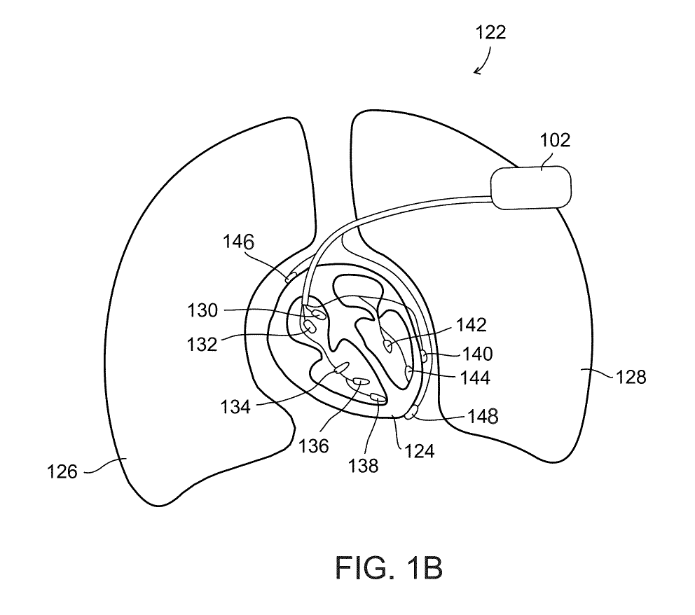

Referring now to the drawings, FIG. 1A illustrates an implanted electrical device such as a cardiac pacemaker 100, which may be used to make impedance measurements for a parametric EIT procedure, according to an exemplary embodiment of the invention. Alternatively the impedance measurements are made using a different implanted device, for example an implantable cardiovertor defibrillator, or a cardiac resynchronization therapy device. Other implantable devices, including implantable devices not used for cardiac therapy, or not used for therapy at all, and including devices that may be invented in the future, may also be used, if they have at least two electrodes in the chest, not necessarily in the heart. Internal electrodes, introduced into the chest temporarily, for example with an endoscope, and not permanently implanted into the chest, may also be used. In general, the methods described herein, that use internal electrodes in the chest, may be performed with any device that puts internal electrodes in the chest, whatever its main purpose.

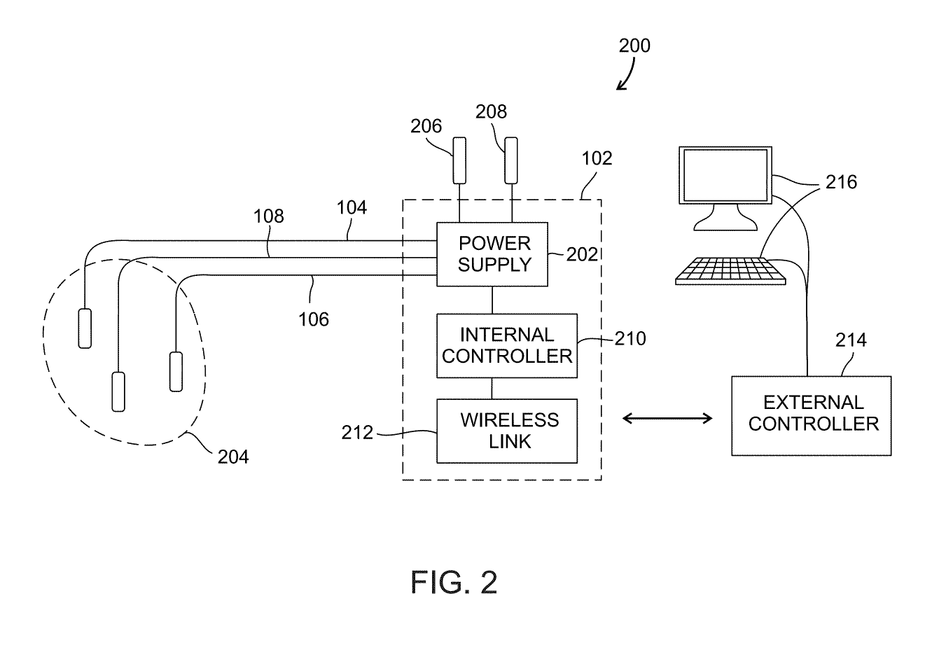

A case 102 contains a power source and control functions, as well as electrodes for injecting current and measuring voltage adjacent to the case, as will be described below in connection with FIG. 2. Leads 104, 106 and 108 connect respectively to electrode 110 located in right atrium 112, to electrode 114 located at left ventricle 116, and to electrode 118, located in right ventricle 120. These electrodes and leads provide electric simulation of the heart as part of the normal pacemaking functions, but can also be used to measure electrical impedance, between different electrodes in the heart or between the electrodes in the heart and electrodes in the vicinity of the pacemaker, in order to perform parametric EIT of the chest, for example in order to measure specific impedance of the lungs, and/or cardiac stroke volume. Optionally, the electric impedance measurements do not interfere with the pacemaking functions of the pacemaker, even if the currents and voltages are of the same magnitude, because the impedance measurements are made at a much higher frequency, for example at 10 kHz or higher, while the pacemaking pulses are essentially DC. Optionally, these electric impedance measurements are made only between the electric stimulation pulses provided for pacemaking purposes, so that the pacemaking voltage and current does not interfere with the impedance measurements, and optionally currents and voltages used for the impedance measurements are small compared to the currents and voltages used for the pacemaking functions, so that the impedance measurements do not affect the functioning of the heart. The voltages and currents used for the impedance measurements are optionally great enough to provide a reasonably high signal to noise ratio for the impedance measurements, for example at least 10 dB, at least 20 dB, at least 40 dB, at least 60 dB, or at least 80 dB.

FIG. 1B is a schematic view 122 of case 102, the heart 124, and lungs 126 and 128, showing alternative locations for the electrodes, in an exemplary embodiment of the invention. It should be understood that generally, all of these alternative electrode locations will not be used at the same time. Right atrium electrode 130 is pressed against the wall of the right atrium. Alternatively, right atrium electrode 132 is suspended in the middle of the right atrium. Electrode 134 is located in the middle of the lead going to right ventricle electrode 136, suspended in the middle of the right ventricle, or right ventricle electrode 138, pressed against the wall of the right ventricle. In general, additional electrodes can be added in the middle of leads going to other electrodes. Optionally, there are separate leads going to electrode 134, and the right atrium electrode, either 136 or 138, and electrode 134 can have a different voltage than the right atrium electrode, and one of them can be used to inject current even if the other is not being used to inject current. Alternatively, only a single lead goes to electrode 134 and the right atrium electrode, and they are in effect a single electrode, but spatially spread out at two location. Left ventricle electrode 140 is located on the outside of the heart, against the wall opposite the left ventricle. Alternatively, a left ventricle electrode 142 may be located inside the left ventricle, suspended in the middle, or pressed against the inner wall, as with left ventricle electrode 144. Alternatively, a right ventricle electrode may be located on the outside of the heart, adjacent to the right ventricle. In some embodiments of the invention, electrodes are placed on the outside of the heart on top, as in electrode 146, and/or on the bottom, as in electrode 148. This may be true, for example, when the implanted device is a defibrillator. Electrodes may also be located in the esophagus, optionally introduced endoscopically rather than implanted. In general, the location of the electrodes may depend on the main function of the implanted or introduced device, and impedance measurements may be done with wherever the electrodes are located, even if the locations are not optimized for impedance measurements.

FIG. 2 shows a block diagram of an EIT system 200, that includes the cardiac pacemaker shown in FIG. 1A, as well as an external controller and user interface in communication with the pacemaker, according to an exemplary embodiment of the invention. Case 102 includes a power supply 202, capable of supplying sufficient current for the stimulation pulses used for pacemaking, and capable of controlling the current sufficiently precisely, and of making sufficiently precise measurements of voltage, to provide accurate measurements of impedance, for the generally much lower currents generally used for the impedance measurements. Typically, for safety reasons, the power supply injects a current of known amplitude between selected electrodes, while measuring the resulting voltage. Alternatively, the power supply applies a known voltage between selected electrodes while measuring the resulting current, or applies a known combination of voltage and current while measuring a different combination of voltage and current, but in all cases, for safety reasons, there is preferably a limit on the magnitude of the current that the power supply is capable of injecting.

Also for safety reasons, the injected current, at least for the impedance measurements, is preferably AC current of a high enough frequency to reduce the risk of harm if the injected current is higher than expected due to a malfunction of the pacemaker. For example, 10 kHz or 20 kHz or 50 kHz is typically used. Optionally, the frequency is low enough so that the current is largely limited to the fluid outside the cells, and relatively little current travels through cell membranes and across cells. For example, the frequency is lower than 100 kHz, for example between 100 kHz and 70 kHz, or between 70 kHz and 50 kHz, or between 50 kHz and 30 kHz, or between 30 kHz and 20 kHz, or between 20 kHz and 10 kHz, or below 10 kHz. Alternatively, frequencies above 100 kHz are used. By keeping the current confined largely to the extracellular fluid, the impedance measurements are more sensitive to the amount of fluid in the lungs, and to the volume of blood in the left ventricle.

Power supply 202 is connected by leads 104, 106 and 108 to electrodes inside heart 204, as shown in FIG. 1A. Power supply 202 is also connected to one or more electrodes located on or near case 102, for example two case electrodes 206 and 208. Optionally, at least one of the case electrodes is incorporated into the covering of the case, for example the covering consists of metal that completely surrounds the case, and the covering itself constitutes one of the case electrodes. Optionally, the case is not completely surrounded by metal, but a part of the covering consists of an insulating material such as a hard bio-compatible plastic, separated two metallic portions of the covering, which constitute two different case electrodes. Optionally, one or more of the case electrodes is located a short distance outside the case, in contact with tissue surrounding the case, for example the left pectoral muscle. If there is more than one case electrode, then they are electrically isolated from each other, or can be electrically isolated from each other for at least some impedance measurements. A potential advantage of having at least two case electrodes is that one of the case electrodes can be used to inject current between the vicinity of the case and one of the heart electrodes, while the other case electrode can be used to measure the resulting voltage between the vicinity of the case and a different one of the heart electrodes. In that way, no electrode using to inject current for a given impedance measurement is also used to measure voltage for that impedance measurement. Using a same electrode for injecting current and measuring voltage can cause inaccuracies or poor signal to noise ratio in the measurement, since the measured voltage may be dominated by a thin high impedance region, with impedance that is possibly not well characterized and/or not predictable over time, adjacent to the electrode being used to inject current, due to poor electrical contact between the electrode and surrounding tissue. Another potential advantage to having at least two electrodes adjacent to the case, particularly if the two electrodes are spatially separated on different sides of the case, is that the two electrodes can be used in different measurements to measure impedance along spatially different paths between the vicinity of the case and the heart electrodes, providing more data and more degrees of freedom for finding parameters such as specific impedance of the lungs, and dimensions of the left ventricle, when performing parametric EIT.

Power supply 202 is connected to an internal controller 210, also located inside pacemaker case 102. Internal controller 210 at least provides control for the autonomous pacemaking functions of the pacemaker, and provides a communications channel, via a wireless communications link 212, to an external controller 214, located outside the body. For example, internal controller optionally communicates voltage data measured by power supply 202 to external controller 214, to use in a parametric EIT calculation, and internal controller 210 optionally controls power supply 202 to inject current and to measure voltage between different pairs of electrodes as requested by external controller 214, according to the requirements of a particular parametric EIT algorithm. In some embodiments of the invention, internal controller 210 performs many or all of the parametric EIT calculations, and optionally even decides autonomously which parametric EIT procedures to perform, with external controller 214 providing only the results to a user, and communicating to internal controller 210 user requests to initiate the parametric EIT procedures, via a user interface 216. In other embodiments of the invention, the parametric EIT calculations are performed largely or entirely by external controller 214, and internal controller 210 communicates the raw current and voltage data from power supply 202 to external controller 214, and communicates to the power supply requests from external controller 214 to inject current to make voltage measurements between particular pairs of electrodes at a given time.

Wireless communications link 212 optionally communicated between internal controller 210 and external controller 214 by radio waves, or by coils that are inductively coupled across the skin of the patient, or by any other known method to communicate between an outside user and an implanted medical device. Although communications link 212 could also use a wire that penetrates the patient's skin, using a wireless link has the potential advantage of making infections less likely. User interface 216 optionally includes one or more of such standard pieces of equipment as a keyboard, a mouse, a display device such as a monitor, and a printer.

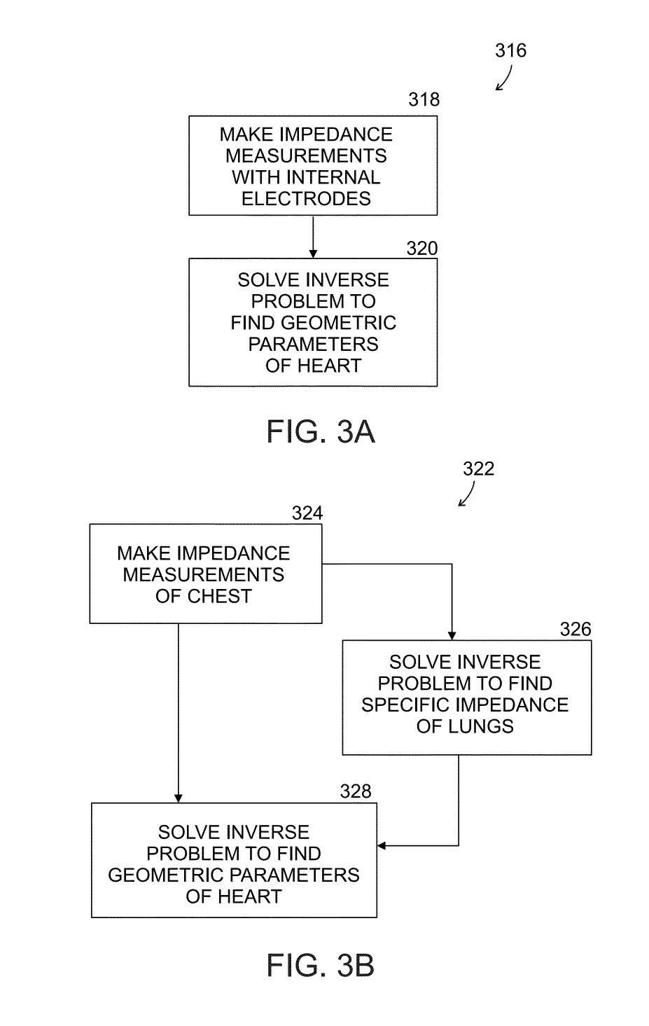

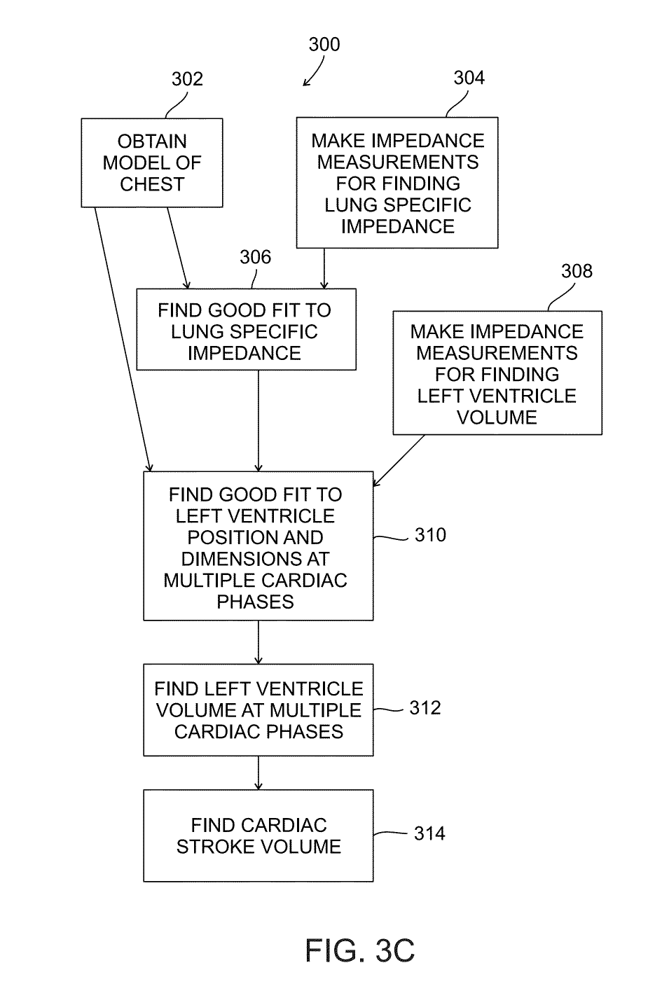

FIG. 3A shows a flowchart 316 with an overview of a method of finding geometric parameters of the heart by parametric EIT, using system 200, in an exemplary embodiment of the invention. At 318, impedance measurements are made with internal electrodes. At 320, the inverse problem of Poisson's equation is solved, to find geometric parameters of the heart that would give a good fit to the measured impedances found at 318, for an electrical model of at least part of the body.

FIG. 3B shows a flow chart 322, similar to flowchart 316, but with the impedance measurements, made at 324, also used to find specific impedances of the lungs, at 326, in an exemplary embodiment of the invention. The specific impedances of the lungs are then used as part of the electrical model of the body, to find values of the geometric parameters of the heart, at 328. Alternatively, a single inverse problem is solved, with both the specific impedances of the lungs and the geometric parameters of the heart, as free parameters, to find all of them together, self-consistently. However, the geometric parameters of the heart have little effect on the specific impedances of the much bigger lungs, so it may be simpler, and cause little loss of accuracy, to first find the specific impedances of the lungs, and then find the geometric parameters of the heart.

FIG. 3C shows a flowchart 300 similar to flowchart 322, but with further steps of a method for using parametric EIT to find a cardiac stroke volume of a patient, in an exemplary embodiment of the invention. At 302, an electrical model of the chest is obtained. Optionally, the model is two-dimensional, for example showing the locations of different tissues in a cross-section of the chest, normal to the vertical axis of the patient, going through the ventricles of the patient's heart, as well as the patient's lungs. Alternatively, a three-dimensional model of the chest is used, including the entire heart and lungs, and other organs, muscles, bones, and blood vessels. Optionally the model is not limited to the chest, but includes the entire torso, including the abdomen, and optionally it includes other parts of the body, including one or more of the neck, the head, the arms, and the legs. Whether the model is two-dimensional or three-dimensional, it includes specific impedance values for different tissues shown in the model.

“Specific impedance,” as used herein, means the resistivity of the tissue, plus any reactive part of the impedance for that tissue, at the frequency at which the impedance measurements are being made, for example at 10 kHz. Specific impedance is a property of the tissue, independent of its shape, and has the dimensions of ohm-meters. Specific impedance should not be confused with impedance, which has the dimensions of ohms, and is the result of making an impedance measurement between a pair of electrodes, or between two pairs of electrodes, one used for injecting current and one used for measuring voltage. The results of an impedance measurement depend on the specific impedance of tissues in the general region of the body where the measurement is made, as well as on the spatial distribution of those tissues, as given for example in a model of the chest. In EIT (electric impedance tomography), a plurality of impedance measurements between electrodes at different locations are used to find at least some aspects of the specific impedance and/or the spatial distribution of different tissues, by solving an inverse problem.

In some embodiments of the invention, the model includes not only a fixed map of the spatial distribution of different tissues and their specific impedance, but also a dependence of the spatial distribution of tissues, and the specific impedance, as a function of cardiac phase. In the case of a three-dimensional model, this is sometimes referred to in the literature as a “4-D” model, with the fourth dimension being time, or cardiac phase. For example, at a cardiac phase at which a tissue has a greater degree of blood perfusion, it will typically have a lower specific impedance, than at a cardiac phase at which there is a lower degree of blood perfusion in that tissue. Using a three-dimensional model has the potential advantage that it may give more accurate results than a two-dimensional model, but it requires more intensive calculations that using a two-dimensional model. Using a model that depends on cardiac phase has the potential advantage that it may provide more accurate results than a model that does not depend on cardiac phase, but requires more computer memory.

Optionally, the model is based on data for the patient being examined, for example it is based on a CT scan and/or a magnetic resonance image obtained for that patient. Alternatively, the model is based on a typical or average patient, optionally a typical or average patient with one or more characteristics matching the patient being examined, for example gender, age, and body mass index. Using a model that is based on data for the same patient has the potential advantage of providing more accurate results, especially if the impedance measurements for the parametric EIT procedure are made when the patient is in the same position as when the CT scan or MRI was made, for example in a supine position, so that the organs are likely to be closer to the same locations than if the patient were in a different position, and if the images were made relatively recently. However, such a patient-specific model may not always be available, and even if it is, there may be changes in the shape and/or positions of the organs by the time the impedance measurements are made.

Optionally, the impedance measurements are done at a particular phase of the breathing cycle, for example with the lungs emptied, and the model is based on the same phase of the breathing cycle. Using the phase where the lungs are emptied has the potential advantage that it may be more reproducible and stable than using a different phase, for example when the lungs are full, or in the middle of inhaling or exhaling. Optionally the patient holds his or her breath while the impedance measurements are made. Optionally, the impedance measurements are made quickly enough that there is no need for the patient to hold his or her breath, or the impedance measurements are gated to the breathing cycle over more than one breathing cycle.

At 304, impedance measurements are made for the purpose of finding specific lung impedance, by injecting current between chosen pairs of electrodes. Although current could be injected between more than two electrodes at once, the resulting voltage is expected to be a linear combination of the voltages seen by injecting current between different pairs of electrodes, so no additional information on impedance is expected to be gained that way. The magnitude of current injected is chosen to be low enough to be safe, but to be high enough to produce reasonably accurate measures of impedance with relatively low noise, for example a current of 1 mA, 2 mA or 3 mA, at 10 kHz, 20 kHz, or 50 kHz has been found to work well. Greater, smaller, or intermediate currents and frequencies are also optionally used. The set of electrodes chosen for these impedance measurements is optionally governed by making impedance measurements that can distinguish well between the specific impedance of the left and right lungs. In some prior art systems that use electric impedance measurements to estimate lung impedance, such as the system described by published patent applications US2004/0102712 and US2005/0124908 to Belalcazar et al, and paper by Belalcazar et al cited above, measurements with pacemaker electrodes can provide a relative estimate of left lung impedance as it changes over time, but are very insensitive to right lung impedance. However, the inventors have found that, by using parametric EIT, with a proper choice of electrodes used for impedance measurements, measurements of the specific impedance of the both the left and right lungs can be obtained using internal electrodes alone, and not just relative estimates of changes in impedance over time, but absolute measurements of specific impedance of each lung at a given time. For example, the inventors have found, based on a simulation, that it is particularly useful to inject current between the right ventricle electrode and a first one of the case electrodes, while measuring voltage between a second case electrode and the right atrium electrode, as well as between the second case electrode and the left ventricle electrode. This provides two independent impedance measurements, which can be used to find the specific impedances of the left and right lungs. Although the voltage measurements can be made using the same case electrode that is used to inject current, better results are potentially obtained using a different case electrode to measure voltage, for the reasons discussed above.

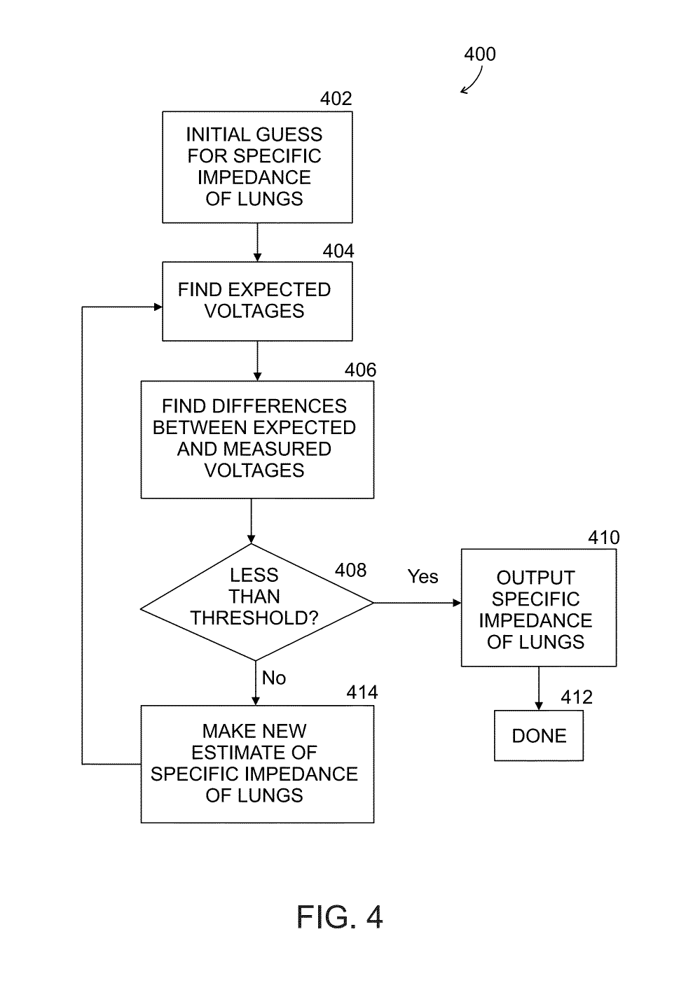

At 306, the inverse problem is solved, i.e. a best fit, or at least a good fit, is found for the left and right lung specific lung impedances that would predict voltages that are close to the measured voltages, for the injected current used. Further details of how this is done are provided below, in the description of FIG. 4. In some embodiments of the invention, it is assumed that the left and right specific lung impedances are the same, and the impedance measurements made in 304 are used to find a best fit, or a good fit, to the single value of specific impedance of the lungs. However, it is potentially advantageous to find the left and right lung specific impedances independently, since they are often different from each other in cardiac patients, and using more accurate values for left and right lung impedance may result in more accurate estimates of cardiac stroke volume. In some embodiments of the invention, it is not assumed that the specific impedance of each lung is uniform throughout the lung, but one or both lungs is divided in two or more regions, and the specific impedance of each region is found independently. The number of independent impedance measurements made is at least equal to the number of regions whose specific impedance is being determined independently. However, especially in the early stages of lung edema, it is often a good approximation to treat the specific impedance of each lung as uniform over that lung, since fluid tends to be distributed fairly uniformly over each lung, in the early stages of lung edema.

At 308, impedance measurements are made, using the pacemaker electrodes, for the purpose of finding the left ventricle volume, by injecting current between chosen pairs of electrodes, and measuring the resulting voltage between the same or a different pair of electrodes. The magnitude of current injected is governed by the same considerations as the impedance measurements made in 304. Optionally, these measurements are made at the same time as the impedance measurements made in 304, and optionally they are the same measurements, used both for determining specific impedance of the lungs, and left ventricle volume. Alternatively, impedance measurements using different electrodes are used for determining the left ventricle volume. If the same set of measurements is used both for specific lung impedance and left ventricle volume, then the number of independent impedance measurements is at least equal to the number of parameters that are being determined independently, for example, left and right lung specific impedances, the dimensions of the left ventricle in three orthogonal directions, and the three coordinates of the center of mass of the left ventricle. Optionally, the impedance measurements used for lung specific impedance are made only at one value of cardiac phase, or only at a few values of cardiac phase, while the impedance measurements used for left ventricle volume are made at many values of cardiac phase. Alternatively, the impedance measurements used for lung specific impedance are made at the same values of cardiac phase as the impedance measurements used for left ventricle volume. The latter procedure may give more accurate results for left ventricle volume, since the lung specific impedances change over a cardiac cycle, due to changes in the perfusion of blood in the lungs, over a cardiac cycle. While the changes in specific lung impedance over a cardiac cycle can also be found from a cardiac-phase-dependent model of the chest, perhaps for a typical patient, with only a base value of lung specific impedance found for the patient being examined, it may give more accurate results to measure the changes in lung specific impedance over a cardiac cycle, for each patient being examined.

At 310, a best fit, or at least a good fit to the impedance measurements, is found for parameters related to the left ventricle volume, for example, in the case of a three-dimensional chest model, the dimensions of the left ventricle in three orthogonal directions, and optionally also the three coordinates of the center of mass of the left ventricle. Optionally one or more parameters related to the orientation of the principle axes of the left ventricle are also fitted to the impedance measurements. Alternatively, the orientation of the principal axes of the left ventricle, and/or the position of the center of mass of the left ventricle, are found from the model of the chest, optionally as a function of cardiac phase. If a two-dimensional chest model is used, then, for example, the parameters related to the left ventricle may be the dimensions of the left ventricle in two orthogonal directions, and optionally also the two coordinates of the center of mass of the left ventricle, and/or a direction of orientation of the principle axes of the left ventricle, in the plane of the two-dimensional model.

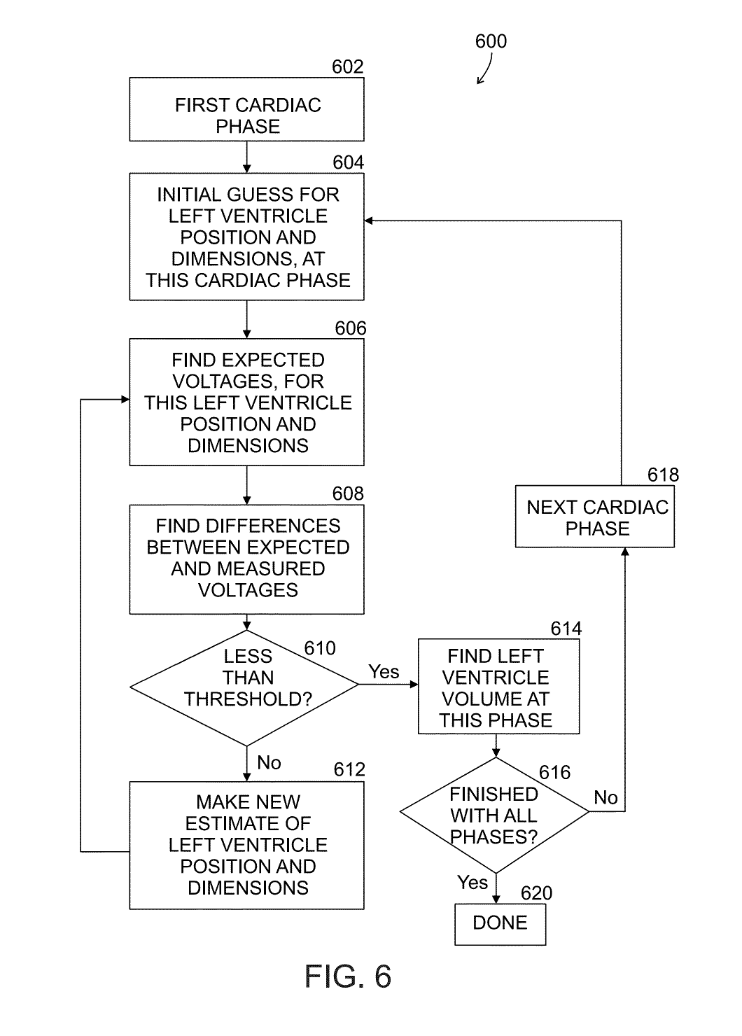

The best fit of the parameters to the impedance measurements is optionally done at each value of the cardiac phase for which the impedance measurements were made at 308. In finding the best fit for the parameters, the specific impedances of the lungs, found at 306, are optionally used. Alternatively, the parameters of lung impedance and the parameters of left ventricle volume are all fitted together to a single set of impedance measurements, for each phase. Further details of how the parameters are found at 310 are provided below, in the description of FIG. 6.

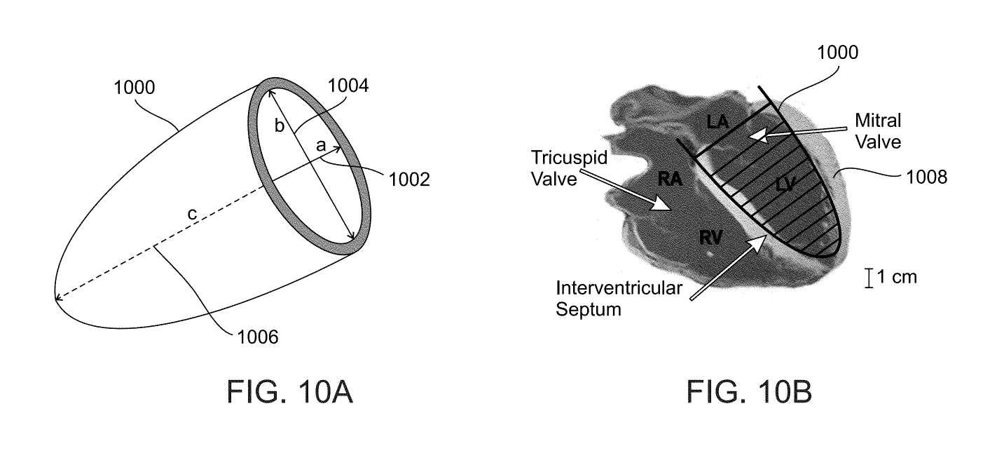

At 312, the left ventricle parameters found at each value of the cardiac phase are optionally used to find the volume of the left ventricle, at each value of the cardiac phase. For example, if the left ventricle is modeled as an ellipsoid or semi-ellipsoid with diameters on three orthogonal principle axes treated as three free parameters of the fit to the impedance measurements, as will be described in more detail below in the description of FIG. 10A, then the volume of the left ventricle may be found as π/6 times the product of the three diameters. Alternatively, the left ventricle volume is found immediately after the left ventricle parameters are found for each value of cardiac phase, in 310, before finding the left ventricle parameters for the next value of cardiac phase. Alternatively, the left ventricle volume is never found at all, but only the left ventricle parameters are outputted at each value of the cardiac phase, if that is all that a user requests.

At 314, the cardiac stroke volume is optionally found from the left ventricle volume as a function of cardiac phase. For example, the cardiac stroke volume is the difference between the maximum left ventricle volume, which occurs at the diastole phase, and the minimum left ventricle volume, which occurs at the systole phase. Alternatively, the left ventricle volume as a function of cardiac phase is smoothed, or fitted to a curve of an expected shape, and the cardiac stroke volume is found from the difference between the maximum and minimum values of the curve. Such a smoothing or fitting procedure may give more accurate values for cardiac stroke, since it may be less sensitive to errors in measurement right at the diastole and systole phases. Another advantage of such a smoothing or fitting procedure is that there may be ranges of the cardiac phase for which impedance measurements cannot be made, or cannot be made accurately, because of interference from the electric stimulation pulses generated by the pacemaker for its pacemaking function, and the smoothing or fitting procedure may provide a good estimate of the left ventricle volume for those values of the cardiac phase. Optionally, the cardiac volume is not calculated, but only the left ventricle volume as a function of cardiac phase is outputted.

FIG. 4 shows a flow chart 400, with further details of fitting the lung specific impedance to the measured impedance values, i.e. solving the inverse problem for lung specific impedance, at 306 in FIG. 3, in an exemplary embodiment of the invention. At 402, an initial guess is made for the specific impedance of each lung, at the frequency at which the impedance measurements are being made, for example 10 kHz. Alternatively, if the both lungs are being treated as having the same specific impedance, then an initial guess is made for that specific impedance, or if one or both lungs are divided into regions which are allowed to have different specific impedances, then an initial guess is made for the specific impedance of each region. The initial guess is based, for example, on a typical or average value, optionally on a typical or average value for cardiac patients with clinical symptoms similar to the patient being treated. If the patient has had the parametric EIT procedure performed in the past, or has had lung specific impedance measured or estimated in some other way, then optionally the initial guess is based on the previously measured values of specific lung impedance for that patient. The specific impedance of other organs and tissues is assumed to have the values given by the chest model.

At 404, expected voltages are calculated, for the electrodes that are used for voltage measurements, for the amount of current injected between the electrodes used for injecting current, using the initial guess for the specific impedances of the lungs, and the values from the chest model for the specific impedances of other organs. This calculation, solving the forward problem, is done using any method known in the art of solving the forward problem of Poisson's equation, ∇(σ∇φ)=−I (where I is the injected current) at internal electrodes where current is injected, and 0 everywhere else, with Neumann boundary conditions σ(∂φ/∂n)=J (where J is the injected current density) at external electrodes where current is injected, and 0 everywhere else, at the boundary of the body. Here σ is the conductivity, including a possible reactive part, i.e. σ⁻¹ is the specific impedance, φ is the potential, and n is a normal to the surface. For example, the potential φ is discretized on a grid using the finite volume method, and the integral form of the equation is solved using the iterative SOR (successive over relaxation) method, as described in S. Abboud et al, “Numerical calculation of the potential distribution due to dipole sources in a spherical model of the head,” Comput. Biomed. Res. 27, 441-455 (1994), and in Rosenfeld, M. et al., “Numerical Solution of the Potential Due to Dipole Sources in Volume Conductors With Arbitrary Geometry and Conductivity,” IEEE Transactions on Biomedical Engineering, July 1996, Vol. 43, No. 7, pages 679-689.

At 406, the difference is found between each measured voltage, and the expected voltage found in 406, for the assumed values of specific impedance. A measure of the differences is calculated, characteristic of the largest difference for all the measured voltages, optionally excluding outliers. For example, the measure is the sum, optionally weighted in some way, of the absolute value squared of all the differences, or the measure is the maximum absolute value of the differences. Optionally, the differences are normalized, for example to the measured voltage, before being used to find the measure of differences, or the measure of differences is normalized, for example to a characteristic measured voltage for all the measurements, or to the square of a characteristic measured voltage, such that the measure of differences is a dimensionless measure of the relative differences between the measured and expected voltages, or the square of the relative differences.

At 408, the measure of differences is compared to a threshold value, chosen, for example, to be small enough so that the procedure provides an accurate measure of specific impedance of the lungs, but not so low that the measure of differences will still be above the threshold when the differences are comparable to the noise in the measured voltages, or to errors in the expected voltages due for example to errors in the chest model, which might result in the measure of differences never getting below the threshold. For example, the inventors have found, based on simulations, that a threshold value of 10⁻³, or 10⁻⁴, or 10⁻⁵, or 10⁻⁶ works well, when the measure of differences is a measure of the square of relative difference in voltage. If the measure of differences is found to be less than the threshold value, then the currently assumed values of specific impedance of the lungs are taken to be accurate, and are outputted at 410, and the procedure ends at 412. If the measure of differences is greater than the threshold value, then a new estimate of the specific impedance of the lungs is made at 414. This is done, for example, by finding the Jacobian matrix, which is the derivative of expected voltages with respect to changes in specific lung impedances. Optionally, the Jacobian is found analytically, as described in U.S. Pat. No. 8,131,354 to Arad (Abboud). Alternatively, the Jacobian is found numerically, by solving the forward problem for slightly different values of specific lung impedance, and numerically finding the first derivatives of the expected voltages with respect to the specific impedances of the lungs. The Jacobian is used to make a new guess for the specific lung impedances that would make the expected voltages equal to the measured voltages, or would make the measure of differences as small as possible, for example by using any known linear extrapolation method, for example the Newton-Raphson method, or a modified Newton-Raphson method, such as the Levenberg-Marquardt method which is more stable. Using the Levenberg-Marquardt method, for example, the new guess for the specific impedances of the lungs is found from

Pₖ₊₁=Pₖ−[Jₖ^{T}Jₖ+λ·diag(Jₖ^{T}Jₖ)]⁻¹*Jₖ^{T}[(φ_{c}(Pₖ)−φm]

Here, Pₖ is a vector with the old values of the free parameters, i.e. the specific impedances of the lungs in this case, after the kth iteration, and Pₖ₊₁ is a vector with the new values of the free parameters, i.e. after the (k+1)th iteration. φₘ is a vector with the measured voltages found in the impedance measurements, and φ_{c} is a vector with the calculated values of the voltages expected from the impedance measurements, based on the values Pₖ of the free parameters. Jₖ is the Jacobian matrix, i.e. the derivative of the expected voltages φ_{c} with respect to the free parameters Pₖ, and the term with λ is a regularization matrix added to the Hessian matrix Jₖ^{T}Jₖ so that it will be better conditioned and its inverse can be found more easily. Alternatively, nonlinear extrapolation methods are used, involving higher order derivatives of the expected voltages with respect to the specific impedances of the lungs. The new guess Pₖ₊₁ for the specific impedances of the lungs is then used to find the expected voltages again at 404, and the loop from 404 to 414 is repeated until the difference between expected and measured voltages is less than the threshold at 408, and the inverse problem is deemed to be solved. If there are more voltages from the impedance measurements, than there are free parameters, then the Levenberg-Marquardt method will in general not lead to a set of free parameters for which the calculated voltages exactly match the measured voltages, but rather will in general lead to a local minimum of the energy function ∥φ_{c}−φₘ∥², and this will tend to be small, less than the threshold, if the model is in good agreement with the patient's actual body, and if the measured voltages do not have too much noise. Optionally, to avoid having the calculation continue forever if this local minimum is not less than the threshold, the calculation ends after it has gone more than a maximum number of iterations, for example 10, or 100, or 1000, or a larger, smaller, or intermediate number.

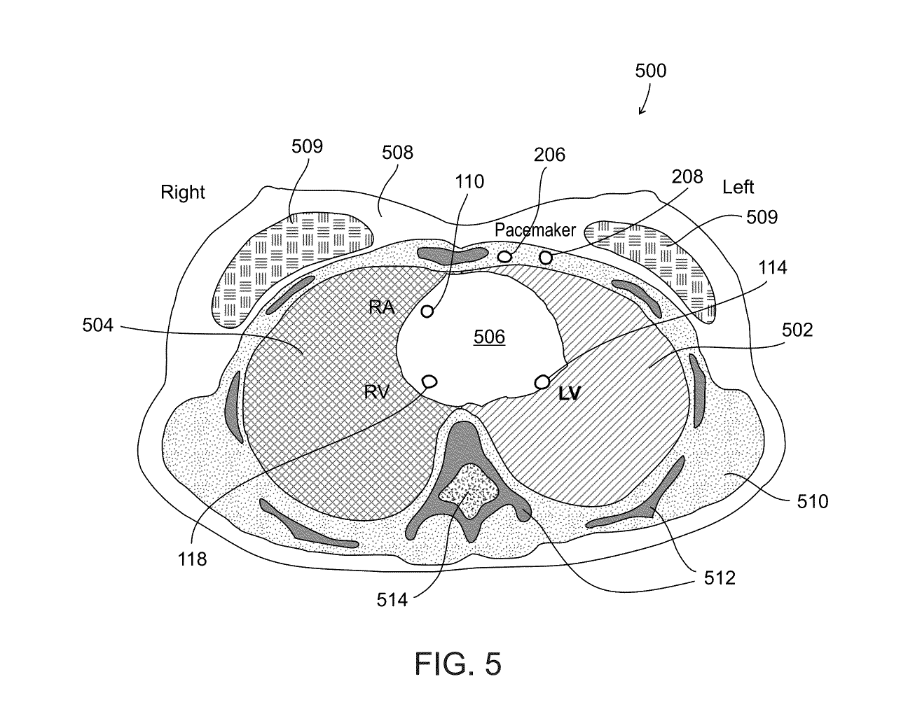

FIG. 5 shows a two-dimensional chest model 500, used in an exemplary embodiment of the invention. The model is divided into regions representing different organs or tissues, and within each region, the specific impedance is assumed to be constant, based, for example, on known typical or average values for other people. The different regions, in this model, are the left lung 502, the right lung 504, the heart 506, the skin 508 (effectively only the dermis, since the epidermis has much higher impedance), breast tissue 509, muscle 510, bone 512, and spinal nerve and marrow 514. Although the heart is divided into chambers (left and right ventricles) and heart muscle tissue, for the purpose of using parametric EIT to find the cardiac stroke volume, optionally, when parametric EIT is only being used to find the specific impedances of the lungs, the heart is treated as having a uniform specific impedance, somewhere in between the specific impedance of blood and the specific impedance of heart muscle. The chest model also includes the location of the pacemaker electrodes, including electrodes 110, 114 and 118 located respectively in the right atrium, the left ventricle and the right ventricle, and electrodes 206 and 208 located at or near the pacemaker case. Table 1 gives exemplary values of specific impedances of the tissues shown in chest model 500, at 10 kHz. The reactive part of the specific impedance may be neglected at 10 kHz, so Table 1 only lists resistivities for these tissues, obtained from C. Gabriel, A. Peyman, and E. H. Grant, “Electrical conductivity of tissue at frequencies below 1 MHz,” Phys. Med. Biol. 54(16), 4863-4878 (2009).

TABLE 1
Resistivities of different tissues

| Tissue | Conductivity @10 KHz [S/m] |
|---|---|
| Air | 0.000 |
| Blood Pool | 0.700 |
| Breast | 0.025 |
| Cardiac Muscle | 0.154 |
| Lungs (deflated) | 0.093 |
| Ribs, Vertebra | 0.020 |
| Skeletal Muscle | 0.340 |
| Skin | 0.003 |
| Spinal Marrow | 0.043 |

FIG. 6 shows a flowchart 600 with details of procedure 310 in FIG. 3C, finding a set of left ventricle parameters that provides a best fit, or at least a good fit, to the results of the impedance measurements made for this purpose, at each of a plurality of different cardiac phases, in an exemplary embodiment of the invention. The procedure, solving the inverse problem for the left ventricle parameters, is initiated at 602, choosing the first cardiac phase to be looked at. At 604, an initial guess is made for the left ventricle parameters that have been chosen as free parameters. For example, the left ventricle is optionally modeled as being a simple geometric shape, for example a semi-ellipsoid, with three free parameters corresponding to the diameter of the left ventricle along each of three principal axes, and three coordinates of the center of mass of the left ventricle, if a three-dimensional chest model is being used, as described above in the description of 310 and 312 of FIG. 3C. Other options for the choice of the free parameters are described above, in the description of 310 of FIG. 3C, and below in the description of FIG. 10A. Optionally, the initial guess for the free parameters is based on values given by the model of the chest, for that cardiac phase. When procedure 604 is repeated later for a different cardiac phase, especially if the cardiac phase only differs by a relative small amount from the previous cardiac phase examined, then optionally the initial guess for the free parameters is based on the final result for the free parameters at the previous cardiac phase examined, or on an extrapolation from two or more previous values of the cardiac phase at which the free parameters of the left ventricle have already been determined.

At 606, expected voltages are calculated, for the electrodes that are used for voltage measurements, for the amount of current injected between the electrodes used for injecting current, using the initial guess for the free parameters of the left ventricle, in this case its position and its dimensions along its principal axes, and using the specific impedances found for the lungs at 306 in FIG. 3, and using the specific impedances of other organs and tissues, and the configuration of organs and tissues, given by the chest model. This calculation, solving the forward problem, is done using any method known in the art of solving the forward problem of Poisson's equation. The calculation is similar to that used in 404 of FIG. 4, for solving the forward problem for the lung specific impedance.

At 608, the differences are found between the expected voltages calculated in 606, and the measured voltages found in 308 of FIG. 3, for this cardiac phase. A measure of the differences is found, characteristic of the largest difference for all the measured voltages, and optionally normalized so that it reflects a relative difference in the measured and expected voltages, using any of the methods described for 406 in FIG. 4.

At 610, it is determined if the differences are less than a threshold. The threshold is optionally chosen according to the considerations described for 408 in FIG. 4. If the measure of differences in greater than the threshold, then a new estimate of the free parameters for the left ventricle, for example the dimensions along the principal axes and the coordinates of the position of the left ventricle, are found at 612. This is optionally done in a similar way to making a new guess for specific impedances of the lungs in 414, but using the free parameters of the left ventricle, instead of the specific impedances of the lungs, as the independent variables. The Jacobian, found analytically or numerically, is the Jacobian of the expected voltages with respect to the free parameters of the left ventricle, rather than with respect to the specific impedances of the lungs. The new guesses for the free parameters of the left ventricle are optionally found by extrapolating the free parameters to find values where the measure of differences between the expected and measured voltages is as small as possible, using a Newton-Raphson method or another extrapolation method, as described for 414. The new guesses for the free parameters of the left ventricle are then used in 604, and the loop from 604 to 612 is repeated until the measure of differences is less than the threshold at 610.

If the measure of differences is less than the threshold, then the free parameters of the left ventricle are accepted as accurate, and the volume of the left ventricle is optionally found, for this value of the cardiac phase, at 614, as described above for 312 of FIG. 3C. Alternatively, the left ventricle free parameters for each value of cardiac phase are stored in memory until all they have been found for all values of cardiac phase, and are then calculated at 312 of FIG. 3C.

At 616, if the left ventricle volume has been found for all of the cardiac phases for which impedance measurements have been made, then the procedure is finished at 620. If there are more cardiac phases for which the left ventricle volume still has to be found, then the next value of cardiac phase is chosen, and a new initial guess for the free parameters is made for the next cardiac phase, at 604. Although the different values of cardiac phase can be used in any order, a potential advantage of using the values of cardiac phase in increasing or decreasing order is that the free parameters of the left ventricle are likely to change relatively little from one value of cardiac phase to the next, so the previously found free parameters can be used as good initial guesses at the next value of cardiac phase, possibly resulting in faster convergence of solving the inverse problem for that value of cardiac phase.

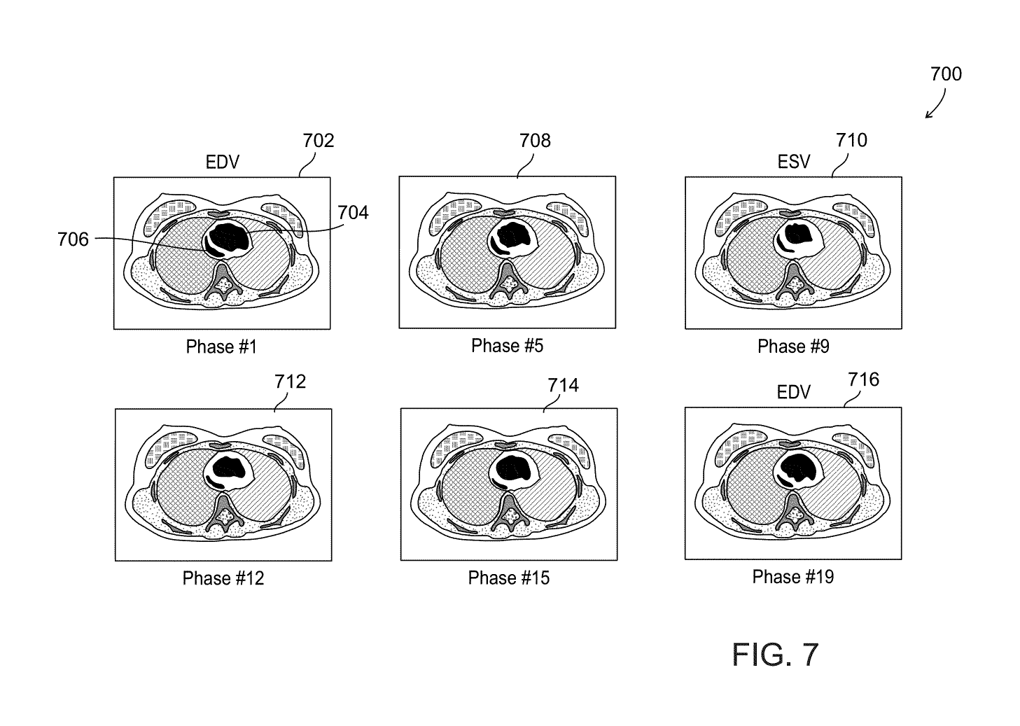

FIG. 7 shows a series 700 of images of the chest, taken over a cardiac cycle, based on manual segmentation of MRI images of an axial cross-section of the chest of a woman, including the left and right ventricles of the heart, from S. Zlochiver et al, “Parametric EIT for Monitoring Cardiac Stroke Volume,” Physiol. Meas. 27, S139-146 (2006), used in an exemplary embodiment of the invention. Images are shown for six different values of cardiac phase, starting with the diastole phase in image 702. The left ventricle 704 and the right ventricle 706 may be seen in each image, separated by and surrounded by heart muscle tissue. The other tissues are the same as those shown in FIG. 5. In image 708, during the rise in blood pressure, the left ventricle contracts, and reaches its minimum volume in image 710, at the systole phase. In images 712 and 714, as the blood pressure falls, the left ventricle expands again, reaching its maximum size at the next diastole phase, in image 716.

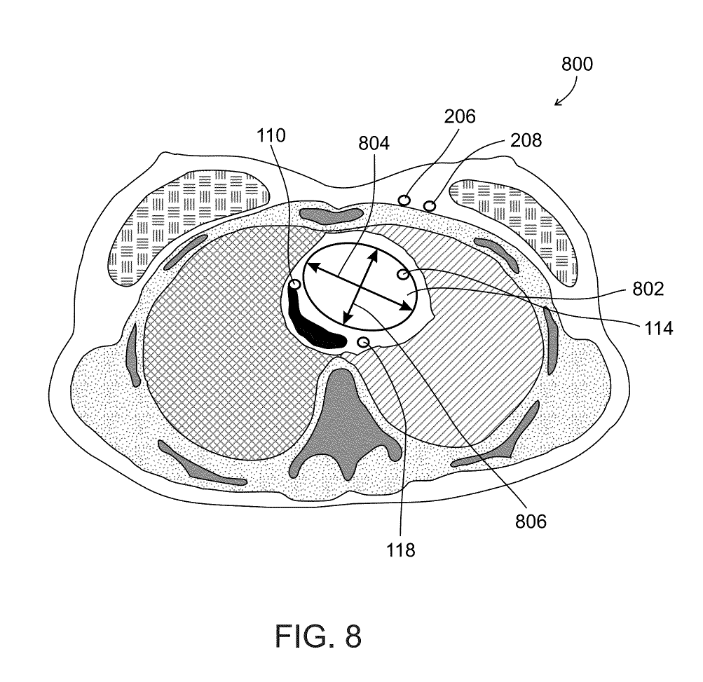

The left ventricle in images 702, 708, 710, 712, 714 and 716 may be approximated as an ellipse, oriented at an oblique angle to the plane of symmetry of the body, with its diameter along its major and minor axes changing as a function of cardiac phase. FIG. 8 shows a two-dimensional model 800 of the chest, based on this observation, that is optionally used for the parametric EIT procedure described in FIG. 3. The model is like the images of the chest in FIG. 7, except that the left ventricle is replaced by an ellipse 802, with major axis 804 and minor axis 806. The locations of pacemaker electrodes 110, 114 and 118, in the right atrium, left ventricle and right ventricle respectively, and electrodes 206 and 208, at two different locations on or adjacent to the pacemaker case, are also shown in FIG. 8, as small white circles. If the model in FIG. 8 is used in the parametric EIT procedure described in FIG. 3C, then optionally the major and minor diameters of ellipse 802 are used as free parameters of the left ventricle. Optionally, one or both of the coordinates of the center of ellipse 802, and/or the angle of orientation of the major axis of ellipse 802, are also used as free parameters of the left ventricle. Alternatively, the coordinates of the center of the ellipse, and/or the angle of orientation of the major axis, are not free parameters, but have constant values based for example on the images in FIG. 7, or have values that are functions of the cardiac phase, based for example on the images in FIG. 7, but are not free parameters in the parametric EIT procedure.

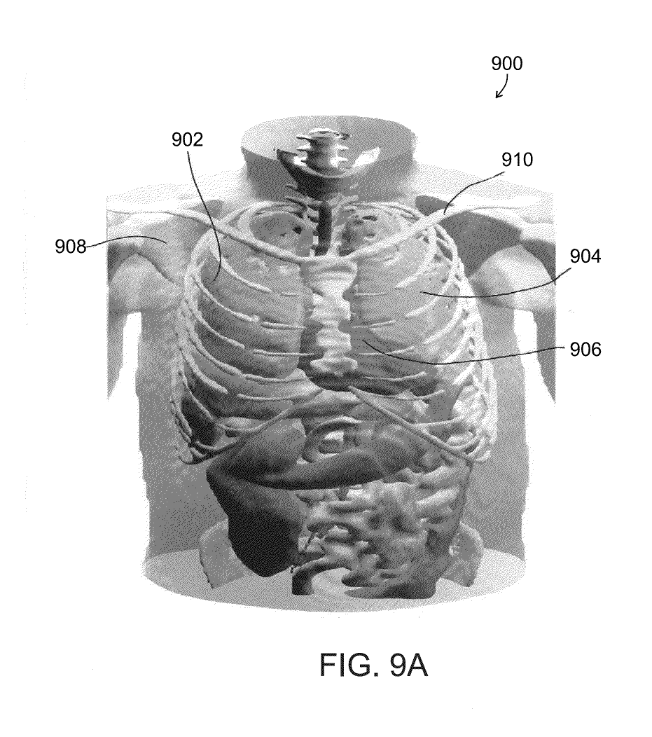

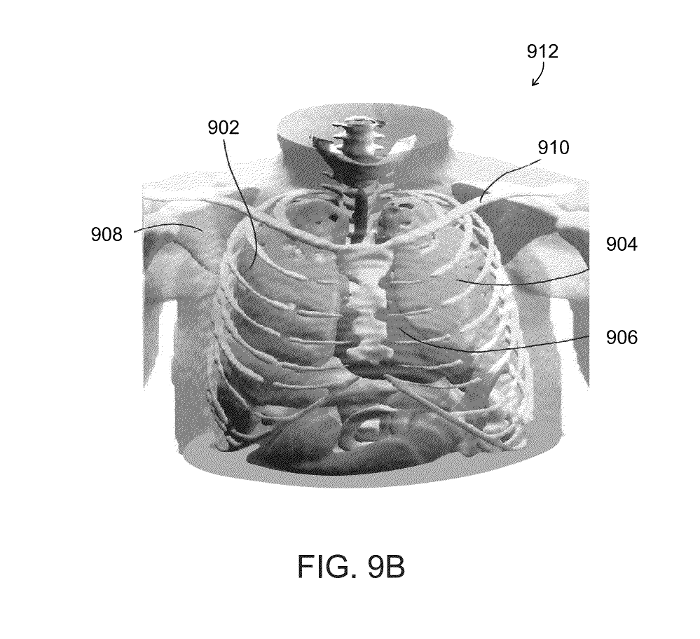

FIG. 9A shows a three-dimensional model 900 of a human torso, that may be used as a model of the chest in the parametric EIT procedure described in FIG. 3, in an exemplary embodiment of the invention. This model, the XCAT (eXtended CArdiac Torso) 4D model described by C. Gabriel, A. Peyman, and E. H. Grant, cited above, includes specific impedance values for 20 different tissues, for frequencies ranging from DC up to 1 MHz, as a function of cardiac phase. Optionally, the model in FIG. 9A is used as a three-dimensional chest model, dependent on cardiac phase, in the parametric EIT procedure described in FIG. 3C.

FIG. 9B shows an alternative three-dimension model 912 of a human torso, not extending as far down into the abdomen as the model in FIG. 9A, that may be used in an exemplary embodiment of the invention.

FIG. 10A shows a cup-like shape 1000, that can be used to model the left ventricle, in the parametric EIT procedure described in FIG. 3C, in an exemplary embodiment of the invention. Cup-like shape 1000 is a semi-ellipsoid, for example, with diameters 2a, 2b, and c, along its principal axes 1002, 1004, and 1006 respectively, used as free parameters in the parametric EIT procedure. The volume of cup-like shape 1000, if it is a semi-ellipsoid, would be 2πabc/3, or π/6 times the product of the three principal diameters. Alternatively, the cup-like shape is a truncated paraboloid, which would have a diameter of πabc/2, or π/8 times the three principal diameters. Alternatively, any other three-dimensional shape, with three free parameters characterizing its dimensions in three orthogonal directions, may be used. If the shape is approximately cup-like, for example if it can be fitted well to a semi-ellipsoid, or to a truncated paraboloid, for some set of principal diameters, for any set of values of its three parameters, at least for a range of principal diameters that are typical of a left ventricle, then it is expected that it would work about as well as a semi-ellipsoid for modeling a left-ventricle in a parametric EIT procedure. Here, “fitted well” means, for example, overlapping in volume by at least 80%, or overlapping by at least 90%, or overlapping by at least 95%.

Even in a three-dimensional model, the left ventricle need not have three free parameters, corresponding for example to a, b, and c in FIG. 10A. Optionally, the left ventricle is modeled as a cup-like shape, for example, but with only one or two free parameters. The third dimension of the cup-like is optionally expressed in terms of the other two dimensions, based for example on an empirical study of how the left ventricle changes in shape as it expands and contracts, leaving only two free parameters. Optionally, both the second and third dimensions of the cup-like shape are expressed in terms of only a single free parameter, based on such an empirical study. Optionally, this is done also with a two-dimensional model where the left ventricle is modeled as an ellipse. Alternatively, the left ventricle is described by four or more free parameters, with the fourth parameter describing some other aspect of its shape, to provide a more realistic model of the left ventricle than the cup-like shape of FIG. 10A.

FIG. 10B shows cup-like shape 1000 superimposed on a left ventricle 1008 in an image of the heart, in a coronal cross-section, showing that it provides a good fit to a real left ventricle. That a semi-ellipsoid provides a good fit to a left ventricle in the third dimension as well, may be seen from FIG. 7, which shows that the left ventricle, in an axial cross-section, provides a good fit to an ellipse. When the left ventricle is replaced by a cup-like shape in a three-dimensional model, or by an ellipse in a two-dimensional model, the original left ventricle, as obtained from the chest or torso model, is filled in with heart muscle tissue, and is replaced by a hollow space that is a cup-like shape, or an ellipse, or whatever shape is being used to model the left ventricle.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find computational support in the following examples.

### EXAMPLES

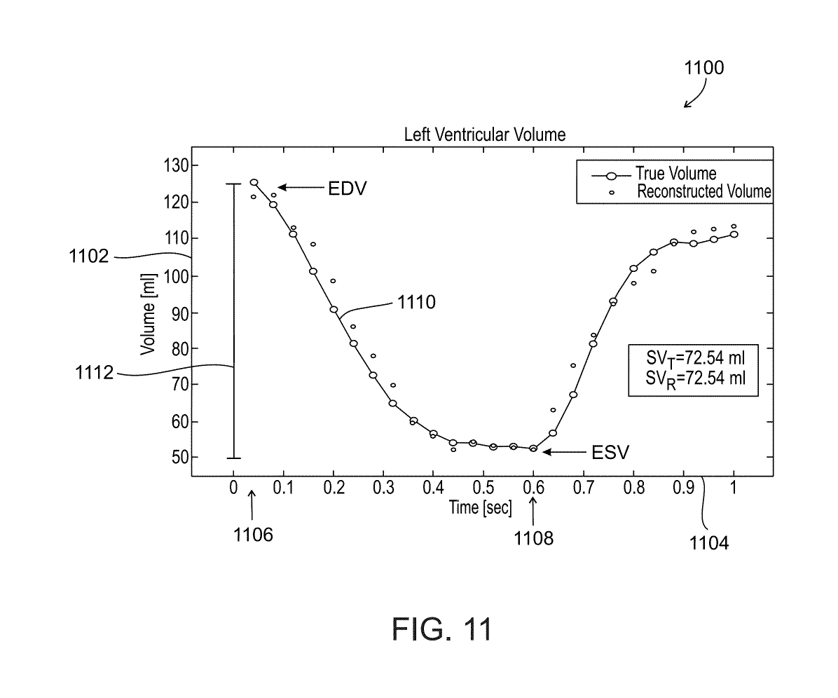

Reference is now made to the following examples, which together with the above descriptions illustrate some embodiments of the invention in a non limiting fashion. FIG. 11 shows a plot 1100 of left ventricle volume, as a function of cardiac phase, as calculated from a simulation of a parametric EIT procedure, using a three-dimensional model of the torso, dependent on cardiac phase, the “XCAT 4D” model described above in FIG. 9A, and compared to the actual left ventricle volume as a function on cardiac phase, in the model. Left ventricle volume, in milliliters, is given by vertical axis 1102, and time t, in seconds, is given by horizontal axis 1104, which extends for 1 second, slightly less than a full cardiac period. The diastole phase 1106 occurs at t=0.03 seconds, and the systole phase 1108 occurs at t=0.6 seconds. Curve 1110 shows the true left ventricle volume from the model, while the left ventricle volume as calculated from the simulated parametric EIT procedure is given by the isolated points on plot 1100. The cardiac stroke volume 1112 is found by taking the difference between the maximum and minimum left ventricle volume, over a cardiac period. The true cardiac stroke volume in the model is 72.54 milliliters, and the cardiac stroke volume calculated by the simulated parametric EIT procedure is 69.51 milliliters, an error of about 4%.

The parametric EIT procedure modeled the left ventricle as a semi-ellipsoid, with the dimensions along three orthogonal axes as free parameters, and the three coordinates of the center of mass of the left ventricle as free parameters, a total of six free parameters. To find the values of these free parameters, at each value of cardiac phase, six impedance “measurements” were made, with the results of the “measurements” calculated from the model, using the actual left ventricle shape, size, and location, not the semi-ellipsoid approximation. The six impedance measurements consisted of injecting current between one of the case electrodes, and one of the heart electrodes, in all six possible combinations. For each pair of electrodes used to inject current, the voltage was measured between the other two heart electrodes. Because there were six free parameters, and six impedance “measurements,” it was generally possible to find a set of the parameters that produced exact agreement between the “measured” and expected voltages, for each “measurement.” The forward problem of Poisson's equation was solved by using a finite volume method, using voxels that were 5 mm on a side, about 160,000 voxels in the torso model. To solve the inverse problem, the Jacobian was found analytically, and a modified Newton-Raphson method was used to make a new guess for the values of the free parameters, at 612 in FIG. 6, at each iteration of solving the inverse problem. The threshold used in deciding when the inverse problem was solved, at 610 in FIG. 6, was 10⁻⁵, expressed as the sum of the squares of the difference in measured and expected voltage, for a current of 1 mA. The specific impedances of the lungs were not found by a simulated parametric EIT procedure, but were values for a healthy subject, given by the model.

It should be understood that this simulated parametric EIT study does not include all of the sources of error that would be present in a real parametric EIT study performed on a patient. In this simulation, the model of the torso is by definition an accurate model of the “patient,” and the errors in the calculated left ventricle volume are due to 1) the fact that the left ventricle is modeled as a semi-ellipsoid in the parametric EIT procedure, and 2) the finite number of elements used in the finite volume calculation solving the forward problem. In an actual parametric EIT procedure performed with a patient, there would be additional errors due to differences between the anatomy of the patient and the model of the chest used, although these differences could be minimized by using a model based on a recent MRI or CT image of the patient. There could also be errors due to differences in the specific impedance of various organs and tissues of the patient, and the values of specific impedance used in the model, based on a typical or average subject. This is especially true of the lungs, and there may remain residual errors in the specific impedances of the lungs even after they are determined by parametric EIT as described in FIG. 4. There could also be errors due to differences in the actual positions of the electrodes, and their positions in the model. There could also be errors due to noise in the voltage measurements, though those errors could be reduced by making repeated impedance measurements, possibly over more than one cardiac cycle.

Some of these sources of error, including differences in the positions of the electrodes, and noise in the voltage measurements, have been included in other simulations, to estimate how much they affect the results in determining cardiac stroke volume. The inventors estimate that, even with all of these sources of error, it should still be possible to measure cardiac stroke volume in a patient to within 10%, absolutely, using parametric EIT. And all of these sources of error should remain relatively stable over time, so the error in measuring changes in cardiac stroke volume over time, in a given patient, should be much smaller than this.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for determining values of a set of one or more geometric parameters of a model including a patient's heart, the method comprising:
a) making impedance measurements of the patient's chest, at least some of the measurements using internal electrodes; and
b) finding values of the set of parameters for the model, which, when the model is evaluated using said values, provide a good fit to results of the impedance measurements.

2. A method according to claim 1, wherein the geometric parameters comprise parameters that determine a volume of the left ventricle, the method also comprising determining the volume of the left ventricle from the values of the set of parameters.

3. A method according to claim 2, wherein the geometric parameters also comprise parameters that determine a position of the left ventricle.

4. A method according to claim 2, wherein making the impedance measurements, finding the values of the set of parameters, and determining the volume of the left ventricle are done for each of a plurality of cardiac phases.

5. A method for determining cardiac stroke volume of a patient, according to claim 4, also comprising finding the cardiac stroke volume from the volume of the left ventricle at each of the cardiac phases.

6. A method according to claim 1, wherein finding the values of the set of parameters comprises:
a) assigning tentative values to the parameters;
b) calculating what results of the impedance measurements would be, using an electrical model of at least part of the patient's body, if the parameters had those values;
c) using the actual and calculated results of the impedance measurements, and a Jacobian of the calculated results with respect to the values of the parameters, to estimate new values of the parameters for which a measure of difference between the actual and calculated results would be smaller;
d) repeating (b) and (c) until values of the parameters are found that provide a sufficiently good fit to the results of the impedance measurements.

7. A method according to claim 6, wherein the electrical model is a two-dimensional electrical model and said set includes at least two parameters.

8. (canceled)

9. A method according to claim 1, wherein the parameters comprise three parameters corresponding to diameters of the left ventricle in three orthogonal directions, and the left ventricle is modeled in the three-dimensional model as a shape whose volume is determined by the three parameters.

10. A method according to claim 1, wherein the electrical model is based on one or more images made of the patient's body.

11-12. (canceled)

13. A method according to claim 1, wherein the internal electrodes comprise three electrodes implanted in the patient's heart and wherein said three electrodes are implanted respectively in the right atrium, in the right ventricle, and in the left ventricle or on a surface of the heart adjacent to the left ventricle, of the patient's heart.

14. A method according to claim 1, wherein said two electrodes are implanted adjacent to and on different sides of the left ventricle.

15-16. (canceled)

17. A method according to claim 1, wherein the internal electrodes are comprised in an implanted therapeutic device and wherein the implanted device is a member of the group consisting of a cardiac pacemaker, an implantable cardiovertor defibrillator, and a cardiac resynchronization therapy device.

18. (canceled)

19. A method according to claim 1, also comprising finding a value of a specific impedance of the patient's lungs, or values of separate specific impedances of the patient's left lung and right lung, that provide a good fit to results of the impedance measurements.

20-21. (canceled)

22. A method according to claim 1, wherein said making comprises
making impedance measurements of the patient's chest using a total of 5 or fewer electrodes; and wherein said model is a three-dimensional model and wherein finding comprises
finding values of the one or more geometric parameters that provide a good fit to results of the impedance measurements, according to said three-dimensional model.

23. A method according to claim 22, wherein the geometric parameters comprise one or more parameters of the patient's left ventricle and comprising finding the volume of the left ventricle from the parameters of the left ventricle.

24. A method according to claim 23, wherein the geometric parameters comprise dimensions of the left ventricle in three directions that are not coplanar.

25-26. (canceled)

27. A method according to claim 1, wherein the model is a three-dimensional model that models the left ventricle as a cup-like shape with the parameters of the left ventricle as free parameters that are fitted to the results of the impedance measurements.

28. A method according to claim 27, wherein the cup-like shape is a truncated ellipsoid or a truncated elliptic paraboloid or a semi-ellipsoid.

29-30. (canceled)

31. A method for determining one or more geometric parameters of a patient's heart, as a function of cardiac phase, the method comprising:
a) at each of a plurality of cardiac phases, making impedance measurements of the patient's chest; and
b) for each of the cardiac phases, finding values of the one or more geometric parameters that provide a good fit to results of the impedance measurements, according to an electrical model of at least part of the patient's body;
wherein the electrical model includes the geometric parameters as free parameters in modeling the heart, models at least one tissue outside the heart as having one or more of a shape, a position, and a specific impedance that varies as a fixed function of cardiac phase.

32-57. (canceled)

58. A system for determining values of a set of one or more geometric parameters of a patient's heart, comprising:
a) a plurality of electrodes implanted in a patient's chest;
b) a power supply configured to pass a known current between a first pair of the electrodes through the chest;
c) a voltage sensor configured to measure a voltage between two of the electrodes, the same as or different from the first pair of electrodes, while the current is passing through the chest; and
d) a controller configured to find values of the set of parameters that provide a good fit to results of one or more measurements of voltage for known currents, according to an electrical model of at least part of the patient's body.

59-62. (canceled)
